(19) United States
(12) Patent Application Publication
(10) Pub. No.: US 2011/0117658 A1
(43) Pub. Date: May 19, 2011

(54) METHOD OF RATIONAL-BASED DRUG DESIGN USING OSTEOCALCIN

(76) Inventors: Daniel S. C. Yang, Brantford (CA); Quyen Hoang, Dundas (CA)

(21) Appl. No.: 13/019,036

(22) Filed: Feb. 1, 2011

Related U.S. Application Data
(63) Continuation of application No. 10/972,690, filed on Oct. 25, 2004, now abandoned.
(60) Provisional application No. 60/562,237, filed on Apr. 15, 2004.

(30) Foreign Application Priority Data
Oct. 23, 2003 (CA) 2446527

(51) Int. Cl. G01N 33/68

(52) U.S. Cl. 436/86

(57) ABSTRACT

The invention relates to a method of identifying a compound that affects osteocalcin activity, comprising obtaining a 3D structure of osteocalcin or a fragment thereof, designing a compound to interact with, or mimic, the 3D structure of osteocalcin or fragment thereof, obtaining the compound, and determining whether the compound affects osteocalcin activity.

### CROSS-REFERENCE TO RELATED APPLICATION

This application is a continuation of U.S. application Ser. No. 10/972,690, filed on Oct. 25, 2004 which claims priority from U.S. provisional patent application No. 60/562,237, filed on Apr. 15, 2004 and Canadian application no. 2,446,527 filed on Oct. 23, 2003, both of which are incorporated herein by reference in their entirety.

### FIELD OF THE INVENTION

The invention relates to the crystalline form of osteocalcin (“OC”). The invention also relates to methods of using the three-dimensional structure of osteocalcin to design and identify candidate compounds that will activate or inhibit osteocalcin activity. The invention also includes compounds identified using the methods of the invention. The invention also includes osteocalcin derivatives that act to inhibit osteocalcin-hydroxyapatite binding. Furthermore, the invention relates to the use of these compounds/derivatives in the treatment of diseases or degenerative conditions resulting from increased or decreased bone resorption/formation and bony metastases of cancers, such as bone metabolic disorders, osteoporosis, breast cancer, prostate cancer, lung cancer and hypercalcemia malignancy.

### BACKGROUND OF THE INVENTION

All bone types consist of mineralized collagen fibrils as their building block. Each fibril is a type I collagen which is made up of three polypeptide chains about 1000 amino acids long. The chains are wound together forming a triple helix. The average diameter of a triple helix is about 1.5 nm and length of 300 nm. In bone, the fibrils are embedded with hydroxyapatite (“HA”) crystals (Ca₁₀(PO₄)₆(OH)₂). These crystals also contain carbonate, magnesium, fluoride, and other impurities.¹ The bone crystals are plate-shaped with average dimensions of 50×25×1.5-4.0 nm.²⁻⁴

Bone undergoes constant turnover. The turnover process involves the break down of bone by osteoclasts (specialized cells that break down bone) and simultaneous rebuilding by osteoblasts (specialized cells that lay down new bone). This process occurs at discrete sites named basic multicellular units (BMUs), which contain the activities of both osteoclasts and osteoblasts, though in different regions of the BMU.⁵ During the turnover process, a number of extracellular proteins are produced. Some of the major bone matrix proteins include type I collagen, proteoglycans, bone sialoprotein, bone morphogenic proteins, osteonectin, osteopontin, and osteocalcin. With the exception of collagen and bone morphogenic proteins, which provide tensile strength and promote differentiation of bone cells respectively, the functions of these proteins are still speculative. Osteocalcin is closely linked to the process of bone mineralization and bone turnover.

Many bone disorders in humans and other mammals are associated with abnormal bone turnover. Such disorders include, but are not restricted to, osteoporosis, Paget's disease, periodontal disease, tooth loss, bone fractures, rheumatoid arthritis, periprosthetic osteolysis, osteogenesis imperfecta, metastatic bone disease, hypercalcemia of malignancy, and multiple myeloma. The most common of these disorders is osteoporosis. Osteoporosis is a skeletal disease characterized by a low bone mass and microarchitectural deterioration of bone tissue, with a consequent increase in bone fragility and susceptibility to fracture. Up to 20% of women over 50 years of age have osteoporosis. Furthermore, bony metastases of cancers, including breast, prostate and lung cancers, cause pain and potentially death. Up to 70% of breast and prostate cancer deaths were characterized by bony metastases. There is currently no cure or successful treatment for cancer metastases to bone. There is a significant need to both prevent and treat osteoporosis and bony cancer metastases as well as other conditions associated with bone metabolism. Bisphosphonates are currently being used to treat osteoporosis and other bone disorders, however, their mode of action is poorly understood. The prevailing belief is that the therapeutic activity of bisphosphonates is due to their ability to inhibit metabolic enzymes and cause cell death. Bisphosphonates consist of two phosphate groups and are structurally analogous to pyrophosphate; therefore, bisphosphonates have the ability to inhibit enzymes that utilize nucleotide trisphosphate and potentially cause cell death. The activity of bisphosphonates in inhibiting metabolic enzymes and causing cell death is considered to be therapeutic, however it is also likely that they have toxic effects that do not contribute to the therapeutic effects.

Tetracyline is an antibiotic that is currently used as a labeling molecule due to its affinity to bone and its fluorescence. Like bisphosphonate, tetracyline is also effective for the treatment of osteoporosis and bony metastatases of cancers. The current belief is that tetracycline's bone therapeutic activity is conferred by binding to the bone surface thereby making the bone surface less fertile to cancer cells. However, this has not been proven.

Osteocalcin is the most abundant non-collagenous protein found associated with the mineralized bone matrix and it is currently being used as a biological marker for clinical assessment of bone turnover. Osteocalcin is a small (46-50 residue) bone specific protein that contains 3 gamma-carboxylated glutamic acid residues in its primary structure. The name osteocalcin (osteo, Greek for bone; Calc, Latin for lime salts; in, protein) derives from the protein's ability to bind Ca²⁺^{₆} and its abundance in bone.^{7, 8} Osteocalcin is also known as “bone gamma-carboxyglutamic acid protein” (BGP) and “vitamin K-dependent protein of bone”. It is distinguished by the presence of 3 gamma-carboxylated glutamic acids (Gla), although some human osteocalcin has been shown to contain only 2 Gla residues.⁹ Because the primary sequence of osteocalcin is highly conserved among species (FIG. 1) and it is one of the ten most abundant proteins in the human body,¹⁰ it is reasonable to infer that the function of osteocalcin is important.

The primary structure of osteocalcin from all species share extensive identity (see table 1), suggesting that its function is preserved throughout evolution. Conserved features include 3 Gla residues at positions 17, 21, and 24, a disulfide bridge between Cys23 and Cys29, and most species contain a hydroxyproline at position 9. The N-terminus of osteocalcin shows highest sequence variation in comparison to other parts of the molecule. Conformational study of osteocalcin by circular dichroism (CD) has shown the existence of alpha-helical conformation in osteocalcin and that addition of Ca²⁺ induces higher helical content.^{6, 11} Two-dimensional nuclear magnetic resonance (NMR) studies of osteocalcin in solution, while structurally inconclusive, revealed that the calcium-free protein was effectively unstructured except for the turn required by the disulfide bridge between Cys23 and Cys29. All the proline residues (Hyp9, Pro11, Pro13, Pro15, and Pro27) were in the trans conformation. Beta-turns are present in the region of Tyr12, Asp14 and Asn26. The hydrophobic core of the molecule is composed of the side chains of Leu2, Leu32, Val36 and Tyr42. The calcium-induced helix is extremely rigid due to, in part, the hydrophobic stabilization of the helical domain by the C-terminal domain.¹¹

Osteocalcin in solution binds Ca²⁺ with a dissociation constant ranging from 0.5 to 3 mM, with a stoichiometry of between 2 and 5 mol Ca²⁺/mol protein.^{6, 12} It has been suggested that osteocalcin binds to hydroxyapatite (Kd≈10⁻⁷ M).⁹ It appears that the Gla residues in osteocalcin are important for its affinity toward Ca²⁺. Binding of Ca²⁺ induces normal osteocalcin to adopt the alpha-helical conformation; however, thermally decarboxylated osteocalcin showed higher alpha-helical content than normal osteocalcin and the calcium induced alpha-helical formation is lost.⁶

Decarboxylated osteocalcin also lost its specific binding to hydroxyapatite.^{9, 13} When bound to hydroxyapatite, the Gla residues are protected from thermal decarboxylation.⁹ Furthermore, osteocalcin synthesized in animals treated with warfarin, which inhibits the formation of Gla, failed to bind to bone.¹⁴⁻¹⁶ Fourier-transform infrared (FT-IR) spectroscopic studies have shown that the Gla residues in osteocalcin coordinate to Ca²⁺ in the malonate chelation mode, where a Ca²⁺ interacts with two oxygen atoms, one from each of the two COO— groups of a single Gla residue.¹⁷ The binding affinity of osteocalcin for hydroxyapatite increased fivefold by the addition of 5 mM Ca²⁺.⁶ Furthermore, hydroxyapatite competition studies demonstrated that prothrombin (10 Gla/molecule) and decarboxylated osteocalcin fail to compete with ¹²⁵I-labeled osteocalcin bound to hydroxyapatite.¹³ Combining all the information discussed above, a structural model has been constructed.¹³ This model consists of two antiparallel alpha-helical domains. The Gla residues are spaced about 5.4 Å apart on one of the helices, which is similar to the interatomic lattice spacing of Ca²⁺ in the x-y plane of hydroxyapatite. It was therefore, predicted that the Gla residues in osteocalcin bind to the (001) plane of hydroxyapatite lattice.^{6, 18}

In addition to osteocalcin's affinity to hydroxyapatite, it has also been shown that the transition of brushite (CaHPO₄.2H₂O) to hydroxyapatite (Ca₁₀(PO₄)₆(OH)₂) is inhibited by very low concentrations of osteocalcin.¹³ The first in vivo indication of osteocalcin involvement with the mineralization of bone was demonstrated by Hauschka et. al. that osteocalcin appears in embryonic chick bones coincident with the onset of mineralization.¹⁹ Studies of bone physiology in animals maintained on warfarin, which inhibits vitamin K-carboxylase, further supports the importance of osteocalcin in bone mineralization. Rats maintained on warfarin during 8 months showed a dramatic closure of the epiphyseal growth plate, causing a cessation of the longitudinal growth.²⁰ Lambs that were maintained on high doses of warfarin from birth to 3 months of age had a significant decrease of trabecular bone turnover, a decrease of bone resorption, and a dramatic reduction of the bone formation rate.¹⁶ These animal studies suggest that osteocalcin, possibly along with other Gla-containing proteins, is important for bone turnover. Ducy et. al. demonstrated that mineralized bone from osteocalcin-deficient mice was two times thicker than that of wild-type. It was shown that the absence of osteocalcin led to an increase in bone formation without impairing bone resorption and did not affect mineralization.²¹ Ducy et. al. further suggested that osteocalcin may bind to a specific, yet to be identified, receptor to fulfil its function. As a consequence of this suggestion, Bodine et. al. demonstrated that conditionally immortalized human osteoblasts metabolically responded to osteocalcin in solution. Pretreatment of cells with inhibitors of adenylyl cyclase, phopholipase C, and intracellular calcium release inhibited the response of the cells to osteocalcin. It was concluded that these results indicated that osteoblasts express an osteocalcin receptor, and this putative receptor is coupled to a G-protein.²² Evidence for the existence of an osteocalcin receptor on osteoclasts has also been demonstrated. Osteocalcin has been shown to induce chemotaxis, cellular differentiation, and calcium-mediated intracellular signaling in osteoclast-like cells, derived from giant cell tumors of bone.²³

Since information about the functional role of osteocalcin is fragmented and sometimes contradictory, the precise function of osteocalcin and mechanism of action are elusive. The mechanism of osteocalcin's action has been difficult to elucidate due to, in part, the fact that it has no known enzymatic activities. Its activity is apparently conferred only by physical interactions with its target(s), which is undoubtedly dependent on the structural characteristics of osteocalcin. Therefore, a detailed 3D structure of osteocalcin is essential for the understanding of its function and such structure would be of great use in the design and screening for specific modulators, activators or inhibitors of osteocalcin activity. Crystallization of osteocalcin from a fish has previously been reported (Coelho et al. “Crystallization of Osteocalcin from a Marine Fish, Argyrosomus regius.” 9th International Conference on the Crystallization of Biological Macromolecules, Mar. 23-28, 2003, Jena, Germany.) To date, osteocalcin has not been crystallized in mammals. Structural determination of small proteins is rather difficult because (i) heavy atom derivatives tend to destroy the crystal and (ii) another method that involves formation of seleno-methionine, which is commonly used, cannot be used because the host that makes seleno-protein (E. coli) can not make Gla. Therefore, in the case of osteocalcin, crystallizing the protein will not lead to its 3-dimensional structure. Therefore, the reported crystalline form from fish does not equate to obtaining a 3-dimensional structure. As well, protein structural aspects, such as a long or flexible C-terminus or N-terminus, can increase the difficulty of crystallization. The mammalian osteocalcins also have a longer N-terminus and are more difficult to crystallize.

### SUMMARY OF THE INVENTION

The present invention relates to the crystalline form of mammalian osteocalcin. The 3D structure provides a detailed description of osteocalcin's active site and a simple model for its binding to hydroxyapatite. A striking feature of the structure is the ordered arrangement of calcium atoms in the dimer interface. The arrangement of negatively charged residues on the H1 surface is precise for calcium coordination in that the coordination geometries are near perfect. Projection of conserved residues onto the molecular surface of the porcine osteocalcin structure reveals a striking and extensive negatively charged surface centering on helix H1. By docking this surface to the surface of hydroxyapatite, it was shown that the Gla surface of osteocalcin complemented well with the surface of hydroxyapatite. Additionally, when osteocalcin is bound to the surface of hydroxyapatite, other regions including the C-terminus of the protein, which has been shown to possess chemotactic activity,²⁴ would be well oriented to carry out recruitment and signal transduction functions via binding to cell surface receptor(s) on osteoclasts and osteoblasts. Accordingly, the invention includes a crystal comprising osteocalcin, of resolution not less than 1.5 Angstroms. The crystalline osteocalcin preferably has at least one of the following: (i) a conserved surface which is created by atoms from 5 or less metal ions and from the following amino acid residues: Gla17, Gla21, Gla24, Asp30 and Asp34; (ii) a structure comprising three helices, most preferably connected by turns; (iii) a disulfide bridge between Cys23 and Cys29; (iv) Ca2+, wherein osteocalcin comprises amino acid residues Gla17, Gla21, Gla24, Asp30 and Asp34, and wherein Gla17, Gla21, Gla24 and Asp30 coordinate the Ca2+.

Now that the three-dimensional structure of the osteocalcin crystal has been determined, an inhibitor/modulator of osteocalcin-hydroxyapatite binding can be identified through the use of rational drug design by computer modeling with a docking program. This procedure can include computer fitting of potential inhibitors to the osteocalcin-hydroxyapatite binding to ascertain how well the shape and the chemical structure of the potential modulator will bind to hydroxyapatite to compete out osteocalcin. Computer programs can also be employed to estimate the attraction, repulsion, and steric hindrance of the subunits with a modulator/inhibitor. A particular advantage is that selective inhibitors can be identified by comparing the potential inhibitor to the 3D structure of osteocalcin.

The invention includes an isolated and purified molecule comprising a binding surface of osteocalcin defined by the structural coordinates of amino acid residues Gla17, Gla21, Gla24, Asp30 and Asp34 according to Table 3 and/or other binding surface amino acids described in this application. The invention also includes an isolated and purified polypeptide consisting of a portion of osteocalcin starting at amino acid Pro13 and ending at one of amino acids Asn27 to Tyr 46 of osteocalcin as set forth in the pig sequence shown in FIG. 1. Other fragments of osteocalcin and corresponding amino acids in other osteocalcins are also included within the scope of the invention.

The invention also includes an isolated and purified fusion-protein of osteocalcin with serum albumin, which consists of an intact hydroxyapatite binding surface and a sterically impaired cell attachment surface.

The invention also includes an isolated and purified protein having the structure defined by the structural coordinates shown in Table 3. The invention further includes a computer model of osteocalcin generated with the structural coordinates listed in Table 3. Accordingly, the invention also includes a method of identifying a compound that modulates (i.e. increases or decreases) osteocalcin activity, comprising obtaining a 3D structure of osteocalcin or fragment thereof, designing a compound to interact with, or mimic, the 3D structure of osteocalcin or fragment thereof, obtaining the compound, and determining whether the compound affects osteocalcin activity. Mimicking the 3D structure of osteocalcin refers to providing a 3D structure that is similar enough in 3D structure to osteocalcin that it is able to bind hydroxyapatite. Mimicking the hydroxyapatite binding surface of osteocalcin refers to providing a structure that consists of negatively charged atoms at atomic positions less than 1.5 Angstrom RMSD from the location of side-chain carboxylic atoms of osteocalcin residues Gla17, Gla21, Gla24, Asp30 and Asp34 of the disclosed structure or positively charged atoms at atomic positions less than 1.5 Angstrom RMSD from the calcium atoms that are bound to the osteocalcin structure provided. A compound that mimics the 3D structure of osteocalcin or hydroxyapatite binding surface of osteocalcin may be a competitive inhibitor of osteocalcin binding to hydroxyapatite. The designing may be by comparison of a known compound structure or by design (assembly) of a new or known compound structure. The design of the compound preferably interacts with or mimics the conserved surface of the osteocalcin or fragment thereof that binds to the hydroxyapatite crystal.

The 3D structure preferably has at least one of the following: (i) a conserved surface which is created by atoms from 5 or less metal ions and from the following amino acid residues: Gla17, Gla21, Gla24, Asp30 and Asp34; (ii) a structure comprising three helices, most preferably connected by turns; (iii) a disulfide bridge between Cys23 and Cys29; (iv) Ca2+, wherein osteocalcin comprises amino acid residues Gla17, Gla21, Gla24, Asp30 and Asp34, and wherein Gla17, Gla21, Gla24 and Asp30 coordinate the Ca2+.

The method optionally further comprises determining whether the compound interacts with the hydroxyapatite and inhibits osteocalcin activity.

The method preferably further comprises: obtaining or synthesizing the compound, forming hydroxyapatite: compound complex and analysing the complex to determine the ability of the compound to interact with hydroxyapatite. Alternatively, one could create such a complex on a computer and analyze it on the computer.

The method also optionally further comprises:

a) determining the three-dimensional structure of the supplemental crystal with molecular replacement analysis;
b) identifying or designing an inhibitor by performing rational drug design with the three-dimensional structure determined for the supplemental crystal or a fragment thereof.

The invention includes a compound obtained according to a method of the invention.

The invention also includes the use of osteocalcin derivatives to interfere with osteocalcin binding to hydroxyapatite. Such derivatives retain the features on the hydroxyapatite binding surface but change the features on the remaining surfaces such that osteocalcin will not interact with cells/proteins in the original manner. Examples of such derivatives include:

(i) osteocalcin purified from non-human species, including but not limiting to pig, monkey, cow, sheep, goat, dog, cat, rabbit, wallaby, rat, mouse, xenopus, emu, chicken, carp, tetraodon, fugu, bluegill, seabream, swordfish, other fish species, bird species, non-vertebrates
ii) mutating residues in 1-16 and/or 25-end
iii) insertion of residues into 1-16 and/or 25-end
iv) deletion of residues from 1-16 and/or 25-end
v) chemical modification of residues in 1-16 and/or 25-end using standing chemical modification techniques.
vi) crosslinking of osteocalcin to other proteins, peptides or structures.

In another embodiment, only the features on the HA binding surface are altered. This will direct the osteocalcin derivatives to locations other than bone and allow it to compete with the bone-bound osteocalcin in interacting with cellular protein and reduce the recruitment of cell to bone. Such osteocalcin derivative may include, but are not limited to

i) de-carboxylation of Glas by chemical means or by expressing osteocalcin in host cells that cannot synthesize GLA.
ii) Mutation of residues on the surface, as can be deduced from the 3-D structure (including but not limiting to Gla-17, Gla-21, Gla-24, Asp-30, Asp-34), that can cause steric clash with the hydroxyapatite surface and therefore prevent the OC mutant from interacting with the HA surface.

In a further embodiment, bisphosphonate derivatives and tetracycline derivatives may be used to bind the hydroxyapatite surface, thereby inhibiting osteocalcin binding.

Another aspect of the invention includes a method of treating a disease or degenerative condition in a subject, comprising administering to the subject a compound/derivative of the invention or a compound identified with a method of the invention. The diseases or degenerative conditions include those that result from increased or decreased bone resorption/formation and bony metastases of cancers, such as bone metabolic disorders, osteoporosis, breast cancer, prostate cancer, lung cancer and hypercalcemia malignancy.

The invention also includes methods for treating bone disease in a subject comprising administering an effective amount of warfarin, aspirin or deriviatives of either of the foregoing to the subject.

The invention also includes a method for identifying a compound that inhibits osteocalcin activity, comprising
obtaining osteocalcin by recombinant technology, chemical synthesis or purification from bone,
contacting osteocalcin with hydroxyapatite,
adding a test compound to the osteocalcin and hydroxyapatite and determining whether the test compound competes with the bound osteocalcin for hydroxyapatite by measuring the amount of osteocalcin dissociated from hydroxyapatite as a result of the addition of the test compound, wherein a compound that competes with osteocalcin for hydroxyapatite is identified as a compound that inhibits osteocalcin activity.

In the method, the test compound optionally includes fragments of osteocalcin, such as fragments containing Gla17, Gla21 and Gla24 or fragments containing Pro13 to Tyr46 or Pro 13 to Asn27. The osteocalcin is optionally produced by chemical synthesis or recombinant methods and may be produced as a modified osteocalcin molecule. For example, the modified osteocalcin may lack the gamma-carboxylic acids on residues Gla17, Gla21 and Gla24. Test compounds include bisphosphonates and tetracycline as well as a derivative of either of the foregoing.

The present invention also provides a method of treating a bone disease or disorder in an animal, preferably a mammal, such as a human, comprising administering an effective amount of an osteocalcin modulator (activators or inhibitors as described in this application) to the animal.

The invention relates to a method of identifying a compound that affects osteocalcin activity, comprising obtaining a 3D structure of osteocalcin or a fragment thereof, designing a compound to interact with, or mimic, the 3D structure of osteocalcin or fragment thereof, obtaining the compound, and determining whether the compound affects osteocalcin activity. The 3D structure of osteocalcin or fragment thereof optionally comprises a binding site. Designing a compound optionally comprises comparing the structural coordinates of the compound to the structural coordinates of the binding site and determining whether the compound fits spatially into the binding site and modulates, inhibits or activates osteocalcin binding to hydroxyapatite. The 3D structure is optionally determined from one or more sets of structural coordinates in Table 3. The method optionally further comprises introducing into a computer program the structural coordinates described herein (eg. Table 3) defining osteocalcin, wherein the program generates the 3D structure of osteocalcin. The osteocalcin optionally comprises all or part of an amino acid sequence shown in Table 1, and structurally equivalent and structurally homologous sequences having at least 60% sequence identity to a sequence in Table 1. The osteocalcin is optionally isolated from a mammal, preferably a pig or a human. The inhibitor optionally comprises modified osteocalcin. The modified osteocalcin optionally lacks at least one of the gamma-carboxylic acids on residues Gla17, Gla21 and Gla24. The inhibitor optionally comprises a bisphosphonate, tetracycline or a derivative of one of the foregoing. The inhibitor optionally comprises an osteocalcin fragment. The osteocalcin fragment is optionally selected from the group consisting of:
a. Gla17, Gla21 and Gla24;
b. Pro13 to Tyr 46; and
c. Pro13 to Asn27.

The osteocalcin structure optionally comprises the following amino acids in the binding site: Gla17, Gla21, Gla24, Asp30 and Asp34. The osteocalcin optionally comprises a conserved surface with a crystal structure which comprises 5 or less metal ions. The metal ions are optionally calcium. The osteocalcin structure optionally comprises three alpha helices. The helices are optionally connected by turns. The crystalline form of osteocalcin optionally comprises a disulfide bridge between Cys23 and Cys29. The method optionally further comprises: obtaining or synthesizing the compound, forming an osteocalcin:compound complex and analyzing the complex to determine the ability of the compound to interact with osteocalcin. The complex is optionally analysed by X-ray crystallography. The method optionally comprises determining whether the compound inhibits osteocalcin binding to hydroxyapatite with an in vitro or in vivo assay. The method optionally comprises determining whether the compound inhibits osteocalcin binding to hydroxyapatite by determining whether the compound mimics a conserved surface of osteocalcin. Osteocalcin activity is optionally determined by:
a) incubating a test sample comprising osteocalcin, (ii) the compound; and (iii) a substrate comprising hydroxyapatite;
b) detecting osteocalcin binding to hydroxyapatite, wherein reduced binding of osteocalcin to hydroxyapatite indicates that the compound affects osteocalcin activity. The invention also includes a compound obtained according to the methods of the invention.

Another aspect of the invention relates to an isolated and purified molecle comprising a binding surface of osteocalcin defined by the structural coordinates of amino acid residues Gla17, Gla21, Gla24, Asp30 and Asp34 according to Table 3. The invention also relates to an isolated and purified polypeptide consisting of a portion of osteocalcin starting at amino acid Pro13 and ending at one of amino acids Asn27 to Tyr46 of osteocalcin. The invention also relates to an isolated and purified fusion-protein of osteocalcin with serum albumin, comprising an intact hydroxyapatite binding surface and a sterically impaired cell attachment surface.

Another aspect of the invention relates to a computer readable medium with either (a) structural coordinate data according to at least one of the tables recorded thereon, the data defining the three-dimensional structure of osteocalcin, or (b) structural data for osteocalcin, the structural data being derivable from the structural coordinate data of Table 3. The structural coordinate data is optionally obtained by x-ray diffraction with a crystal of the invention. A variation of the invention includes a computer system containing either (a) structural coordinate data according to at least one of the tables, the data defining the 3D structure of osteocalcin, or (b) structural data for osteocalcin, the structural data being derivable from the structural coordinate data of Table 3. The structural coordinate data are optionally obtained by x-ray diffraction with a crystal of the invention.

Another aspect of the invention relates to a method of designing an osteocalcin inhibitor through use of a crystal of the invention or structure coordinates derived therefrom. The invention also includes a method of treating a bone disease in a subject, comprising administering to the subject with at least one of the compounds described herein. The disease is optionally selected from the group consisting of osteoporosis, breast cancer, prostate cancer, lung cancer and hypercalcemia.

Another aspect of the invention optionally includes a crystal comprising mammalian osteocalcin. The invention also relates to a method of preparing an osteocalcin crystal comprising growing the crystal in a reservoir containing 1 to 100 mM of calcium ion, more preferably about 10 mM calcium ion. The calcium ion optionally comprises calcium chloride.

### BRIEF DESCRIPTION OF DRAWINGS

Preferred embodiments are described in relation to the drawings, in which:

FIG. 1. Sequence alignment of Osteocalcin. Protein sequence with the secondary structure elements indicated and the conserved residues highlighted. Positions are identified as conserved if more than 85% of the residues are identical, or similar if hydrophobic in nature. ‘γ’ indicates a Gla residue, open triangles and circles indicate hydrophobic core and Ca²⁺-coordinating surface, respectively. The figure shows sequence corresponding to pig (SEQ ID NO:5), human (SEQ ID NO:3), monkey (SEQ ID NO:1), cow (SEQ ID NO:4), sheep (SEQ ID NO:6), goat (SEQ ID NO:7), dog (SEQ ID NO:8), cat (SEQ ID NO:9), rabbit (SEQ ID NO:2), wallaby (SEQ ID NO:10), rat (SEQ ID NO:11), mouse (SEQ ID NO:12), xenopus (SEQ ID NO:13), emu (SEQ ID NO:14), chicken (SEQ ID NO:15), carp (SEQ ID NO:16), tetraodon (SEQ ID NO:17), fugu (SEQ ID NO:18), bluegill (SEQ ID NO:19), seabream (SEQ ID NO:20), swordfish (SEQ ID NO:21).

FIG. 2. Crystal structure of porcine osteocalcin and the experimental electron density map. Porcine osteocalcin is shown as rod bond model. The solvent-flattened SAS map (contoured at 1.5 sigma) is shown as mesh. Calcium ions are shown as spheres.

FIG. 3. Structure of porcine osteocalcin (SEQ ID NO: 5). a, Protein sequence with the secondary structure elements indicated and the conserved residues highlighted. Positions are identified as conserved if more than 85% of the residues are identical, or similar if hydrophobic in nature. ‘γ’ indicates a Gla residue, open triangles and circles indicate hydrophobic core and Ca²⁺-coordinating surface, respectively. b, Ribbon representation of the crystal structure. The N and C termini are labelled. Side chains of the Ca²⁺ coordinating residues and those involved in tertiary structure stabilization are shown in stick representation. Broken line indicates a hydrogen bond. c, d, Molecular surface representations of porcine osteocalcin. Views in b and c are perpendicular to that in d. e, Crystallographic dimer interface. Spheres and the broken lines represent Ca²⁺ ions and ionic bonds, respectively.

FIG. 4. Model of porcine osteocalcin (SEQ ID NO: 5) engaging an hydroxyapatite crystal based on a Ca²⁺ ion lattice match. Only the best search solution is shown. a, Alignment of porcine osteocalcin-bound and hydroxyapatite Ca²⁺ ions. b, c, Orientation of porcine osteocalcin-bound Ca²⁺ ions in a sphere of hydroxyapatite-Ca lattice (b) and on the hydroxyapatite surface (c). In b, the parallelogram indicates a unit cell; the box approximates the boundary of the slab shown in c and d. d, Docking of porcine osteocalcin on hydroxyapatite. e, Detailed view of d showing the Ca—O coordination network at the porcine osteocalcin-hydroxyapatite interface. Broken lines denote ionic bonds. Isolated spheres and the tetrahedral clusters of spheres represent OH⁻ and PO₄⁻³ ions, respectively.

FIG. 5. Comparison of the top four solutions in the calcium lattice match search. R.m.s.d. in distance between the porcine osteocalcin-bound and the hydroxyapatite calcium ions are 0.44 Å, 0.47 Å, 0.61 Å and 0.61 Å in (i)-(iv). a-d, Refer to legend in FIG. 4(a-d) for the corresponding explanations.

FIG. 6. a, Sedimentation equilibrium analyses in the presence of 10 mM CaCl₂. The best fit is shown as a line through the experimental points, and the corresponding distributions of the residuals are shown above the plots. b, Sedimentation equilibrium analyses in the absence of Calcium. The best fit is shown as a line through the experimental points, and the corresponding distributions of the residuals are shown above the plots.

### DETAILED DESCRIPTION OF THE INVENTION

Porcine osteocalcin crystallized as a crystallographic dimer with a two fold symmetry about the b-axes. There is no direct intermolecular protein-protein interaction within the dimer, but rather, the interactions that hold the dimer together are protein-Ca²⁺-protein. The Gla residues on each monomer are arranged linearly on the protein surface and the row of Ca²⁺ is sandwiched between these two surfaces. The arrangement of the Ca²⁺ atoms is ordered and also has the same 2-fold relationship. The crystal structure POC₁₃₋₄₉ consists of 3 helices, each is separated from another by a turn forming a helix-turn-helix-turn-helix motif. The first helix (H1) spans from Asp17 to Asn26. All three Gla residues lie on one side of H1 helix with their side-chains radiating away from the protein core where they, together with Asp30, coordinate the 5 Ca²⁺ ions that form the dimer interface. The second helix (H2) spans from Asp28 to Asp34. H2 is separated from H1 by a turn between Leu25 and Pro27, which is stabilized by the disulfide-bridge. The turn and the disulfide-bridge position H2 such that Asp30 is oriented correctly for participation in calcium chelation. The third helix (H3) spans from Phe38 to Tyr46. H3 is turned back, via a turn between Ile36 and Phe38, to close proximity of H1 and H2; thereby, forming a hydrophobic core between the three helixes. The hydrophobic core is made up of residues Val22, Leu25, Leu32, Ala33, Ala41, Tyr42, Phe45 and Tyr46. The N-terminus of osteocalcin is flexible.

A striking feature of the structure is the ordered arrangement of calcium atoms in the dimer interface. Such order is characteristic of crystal lattices, showing that the calcium binding surface on osteocalcin is also suited for binding to crystal surfaces. The arrangement of negatively charged residues on the H1 surface is precise for calcium coordination in that the coordination geometries are near perfect. Projection of conserved residues onto the molecular surface of the porcine osteocalcin structure reveals a striking and extensive negatively charged surface centering on helix H1. By docking this surface to the surface of hydroxyapatite the Gla surface of osteocalcin complemented well with the surface of hydroxyapatite. Additionally, when osteocalcin is bound to the surface of hydroxyapatite, other regions including the C-terminus of the protein, which has been shown to possess chemotactic activity,²⁴ would be well oriented to carry out recruitment and signal transduction functions via binding to cell surface receptor(s) on osteoclasts and osteoblasts.

Based on the invention described herein, it is shown that the therapeutic effects of bisphosphonates are conferred by their affinity for bone. The binding of bisphosphonates to bone displaces osteocalcin from the bone surface thereby interfering with the bone turnover process. In this light, new bisphosphonate derivatives should be selected for highest hydroxyapatite binding activity rather than the toxic effect of enzyme inhibition. The hydroxyapatite binding alone should be sufficient to confer therapeutic effects as demonstrated by tetracycline. The invention also shows that tetracyline's bone therapeutic activity is due to the displacement of osteocalcin from the bone surface, thereby removing the adaptor necessary for cancer cell attachment, and preventing cancer cells from attaching to the tetracycline coated/ostecalcin free bone surface. The invention satisfies the need for a way to deliver drugs and other agents exclusively to bone for the treatment of bone disease as well as for bone imaging and diagnostics. Since osteocalcin binds tightly and specifically to bone as disclosed, it is useful as a bone seeking module to carry drugs, proteins, hormones, radioactive atoms and other agents specifically to bone for the treatment of bone disease or bone imaging. For example, 17 beta-estradiol linked to osteocalcin is usefully targeted specifically to bone to treat postmenopausal osteoporosis while minimizing the hormone's effect in other tissues. Anticancer drugs, such as methotrexate, linked to osteocalcin are useful to specifically target bone tumours. ⁸⁹Sr, ¹⁵³Sm, and ¹⁸⁶Re are linked to osteocalcin and administered to a subject for diagnostic and treatment of bone malignancies.

The invention also provides bone and teeth implants that allow rapid integration into the surrounding tissues while minimizing rejection and complications. Since osteocalcin has dual functions of binding to hydroxyapatite as well as recruitment of bone cells, coating of bone implants with osteocalcin promotes rapid integration of the implants. For example, bone and dentine implants are usefully coated with hydroxyapatite which is in turn coated with osteocalcin before implantation which promotes cellular recruitment to the implant surface.

In one aspect the invention is directed to the three-dimensional structure of an isolated and purified osteocalcin and its structure coordinates.

The invention also includes methods of identifying compounds capable of inhibiting osteocalcin binding to hydroxyapatite. The compound should be designed to either mimic or bind to the HA-binding surface on osteocalcin. A compound that mimics the HA-binding surface on osteocalcin would bind to a site on HA that endogenous osteocalcin naturally binds thereby displacing the bound osteocalcin from the bone surface. A compound that complements the HA-binding surface of osteocalcin would bind to osteocalcin and mask the HA-binding surface thereby preventing osteocalcin from binding to bone. (amino acid numbers 14 to 34 in human HA and amino acid numbers 14 to 34 in porcine HA).

Another aspect of the invention is to use the structural coordinates of osteocalcin to homology model other osteocalcin-like species.

This invention provides the first rational drug design strategy for modulating osteocalcin activity. The structure coordinates and atomic details of osteocalcin are useful to design, evaluate (preferably computationally) and synthesize inhibitors of osteocalcin that prevent or treat bone pathologies.

The invention includes methods for identifying compounds that can interact with osteocalcin or the binding site for osteocalcin on hydroxyapatite. These interactions can be easily identified by comparing the structural, chemical and spatial characteristics of a candidate compound to the three dimensional structure of the osteocalcin. Since the amino acids that are responsible for osteocalcin activity and binding were identified by this invention, drug design may be done on a rational basis.

The structure serves as a detailed basis for the design and testing of inhibitors, initially in the computer, but also in vitro in cell culture and in vivo, providing a method for identifying inhibitors having specific contacts with the osteocalcin or an isoform, homologue or mutant or the osteocalcin binding site on hydroxyapatite. The effect of a modification to an inhibitor may be readily viewed on a computer, without the need to synthesize the compound and assay it in vitro. As well, non-protein organic molecules may also be compared to the osteocalcin on a computer. One can readily determine if the molecules have suitable structural and chemical characteristics to interact with, or activate or inhibit, osteocalcin activity. The invention includes the osteocalcin modulators discovered using all or part of an osteocalcin of the invention (preferably the 3D structure) and the methods of the invention.

### Crystals

Crystal Properties

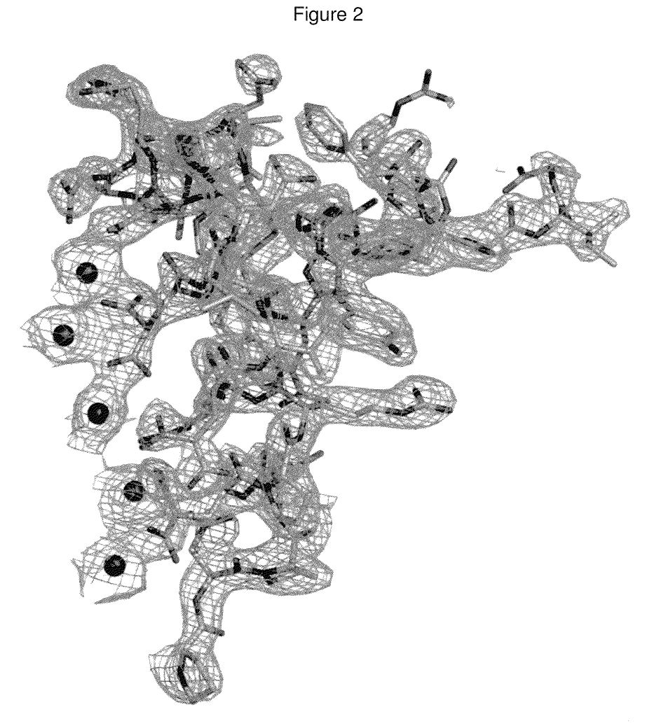

The crystal structure of porcine osteocalcin was determined at 2.0 Å using the Iterative Single Anomalous Scattering method.²⁵ Bijvoet difference Patterson map analysis detected the presence of three tightly bound Ca²⁺ ions and two S atoms corresponding to a disulphide bridge between Cys 23 and Cys 29, which together were used to phase the porcine osteocalcin structure. An atomic model corresponding to residues Pro 13 to Ala 49 was built into well-defined electron density (FIG. 2) and refined to an R_{work} and R_{free} of 25.5% and 28.3%, respectively. Data collection and structure refinement statistics are summarized in Table 2.

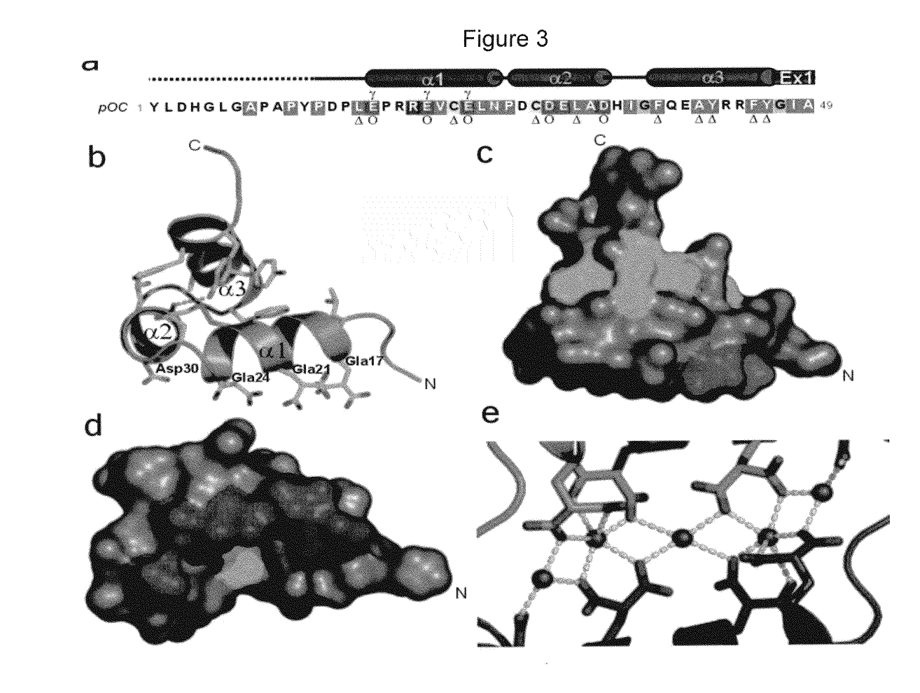

Porcine osteocalcin forms a tight globular structure comprising a previously unknown fold (no matches in the DALI database²⁶ with a topology consisting, from its amino terminus, of three α-helices (denoted α-1-α3) and a short extended strand (denoted Ex1; FIG. 3a). Helix α1 and helix α2 are connected by a type III turn structure from Asn 26 to Cys 29 and form a V-shaped arrangement that is stabilized by an interhelix disulphide bridge involving Cys 23 and Cys 29. Helix α3 is connected to helix α2 by a short turn and is aligned to bisect the V-shape arrangement of helix α1 and helix α2. The three α-helices together compose a tightly packed core involving conserved hydrophobic residues Leu 16, Leu 32, Phe 38, Ala 41, Tyr 42, Phe 45 and Tyr 46. The overall tertiary structure is further stabilized by a hydrogen bond interaction between two invariant residues, Asn 26 in the helix α-1-α2 linker and Tyr 46 in helix α3.

Projection of conserved residues onto the molecular surface of the pOC structure (FIG. 3c, d) shows an extensive negatively charged surface centring on helix α1 (solvent-exposed surface area 586 Å²). Notably, all three Gla residues implicated in hydroxyapatite binding are located on the same surface of helix α1 and, together with the conserved residue Asp 30 from helix α2, coordinate five Ca²⁺ ions (denoted Ca1-Ca5) in an elaborate network of ionic bonds (FIG. 3e). These five Ca²⁺ ions are sandwiched between two crystallographically related porcine osteocalcin molecules and show both monodentate and malonate modes of chelation with extensive bridging.

In the porcine osteocalcin crystal structure, the Ca²⁺ ions coordinated by the Gla residues have an unexpected periodic order reminiscent of a crystalline lattice. Because Gla residues are essential for the interaction of osteocalcin with bone in vivo¹⁶ and for the specific interaction with hydroxyapatite in vitro,⁹ whether the specific atomic arrangement of bound Ca²⁺ ions in the pOC crystal structure mimics the spatial arrangement of Ca²⁺ ions in hydroxyapatite was investigated. To do so, a comprehensive real-space search for a spatial match between the pOC-bound Ca²⁺ ions and the Ca²⁺ ions in crystalline hydroxyapatite (Ca₅(PO₄)₃OH, space group P6₃/m, unit-cell dimensions a=b=9.432 Å, c=6.881 Å)²⁷ was done. Search solutions were ranked by root mean square (r.m.s.) deviations of distances between osteocalcin bound and hydroxyapatite Ca²⁺ ions.

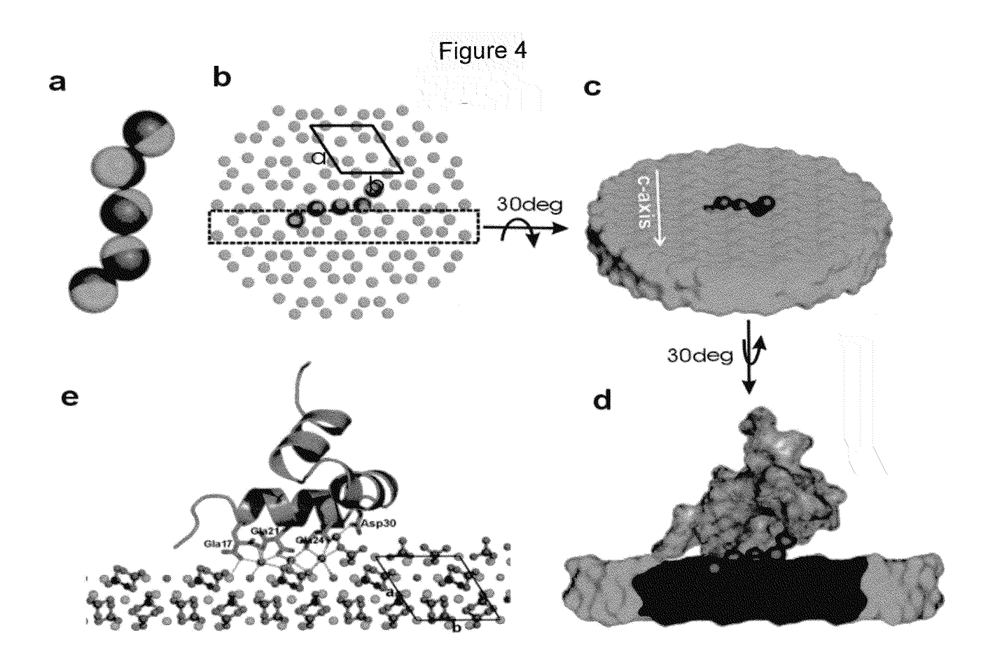

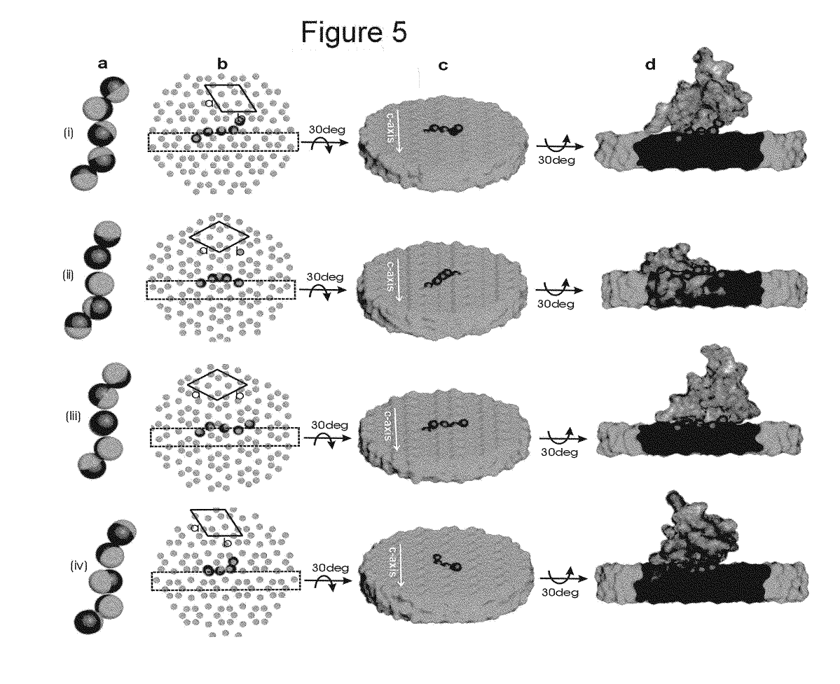

Unique solutions within 1 s.d. (0.29 Å) of the best solution were chosen for graphical analysis. Molecular surfaces of hydroxyapatite defined by the Ca²⁺ ions of the best search solutions were constructed for docking analysis (FIG. 4 and FIG. 5). The best (r.m.s. deviation 0.44 Å) and fourth best (r.m.s. deviation 0.62 Å) solutions in the search correspond to the prism face (100) of HA, whereas the second (r.m.s. deviation 0.47 Å) and third (r.m.s. deviation 0.61 Å) best solutions correspond to the secondary prism face (110). Notably, the prism face is the predominant crystal face expressed in geological²⁸ and synthetic hydroxyapatite²⁹ and, although the predominant crystal face of hydroxyapatite expressed in bone has not been unambiguously determined, atomic force microscopy³⁰ and diffraction analysis⁴ indicate that the expressed face lies parallel to the crystal c axis. The best solution identified in the search also corresponds to a crystal face that lies parallel to the crystal c axis.

Although the best and second best solutions both show good lattice match statistics, only the best solution gives rise to a docking mode of porcine osteocalcin to hydroxyapatite that is free of steric clash. Using the best search solution, a more detailed binding model was generated. The coordination network of Ca—O atoms at the osteocalcin-hydroxyapatite interface closely mimics that in the hydroxyapatite crystal lattice (r.m.s. deviation Ca—O bond distance, 0.19 Å; r.m.s. deviation Ca—O—Ca bond angle, 9.63°; FIG. 4e).

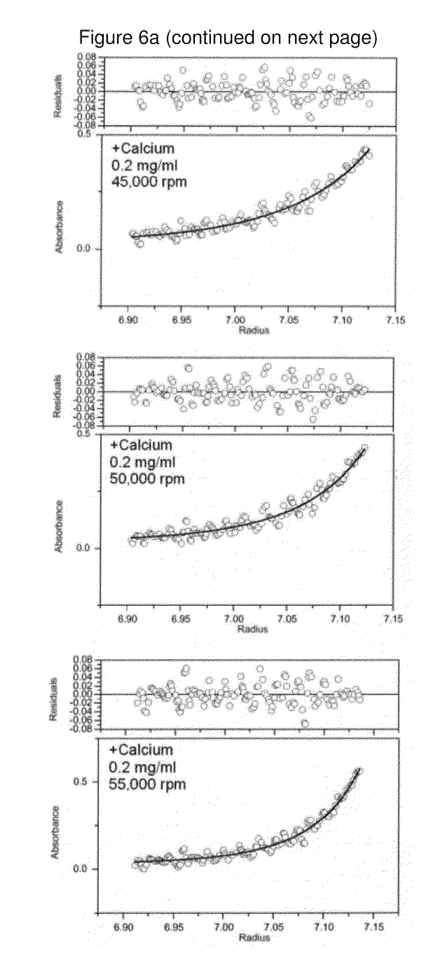

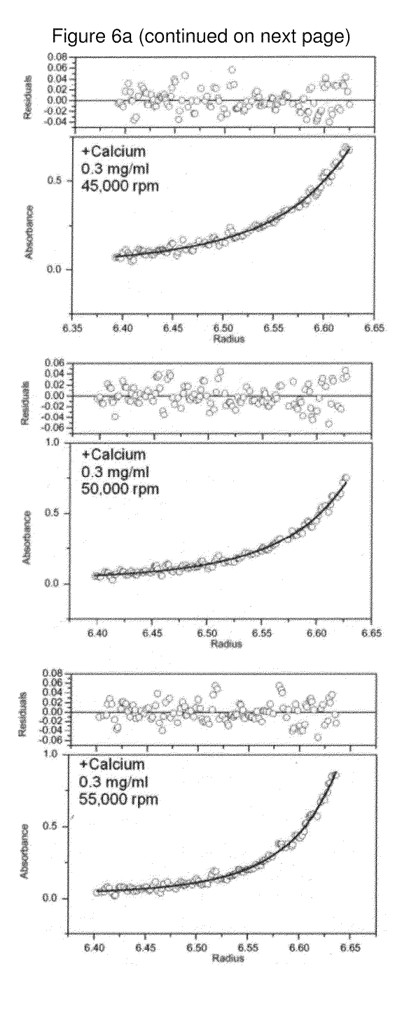

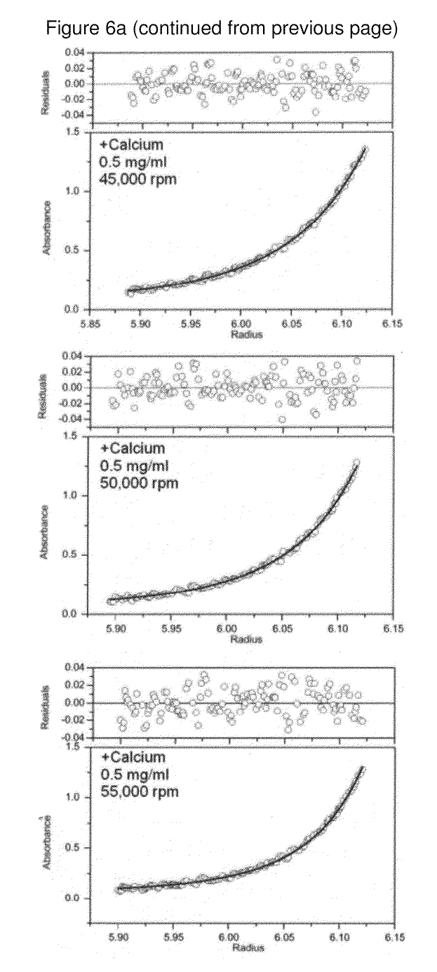

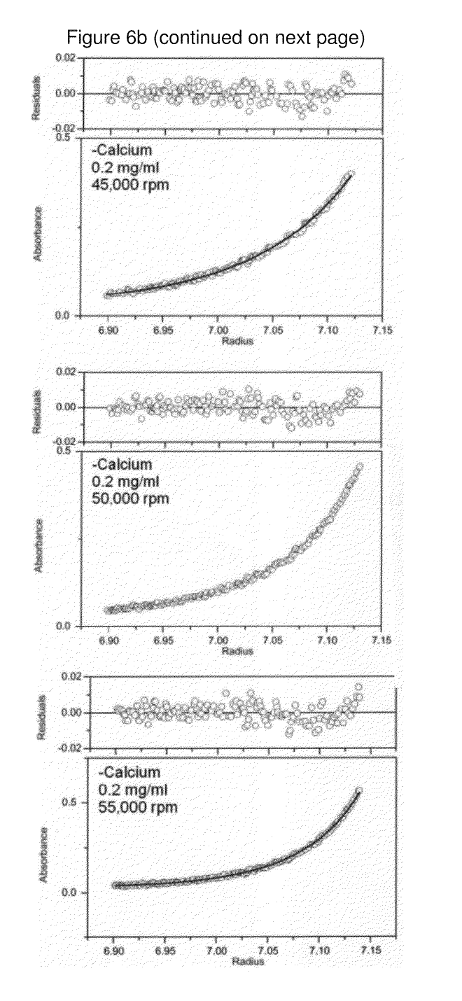

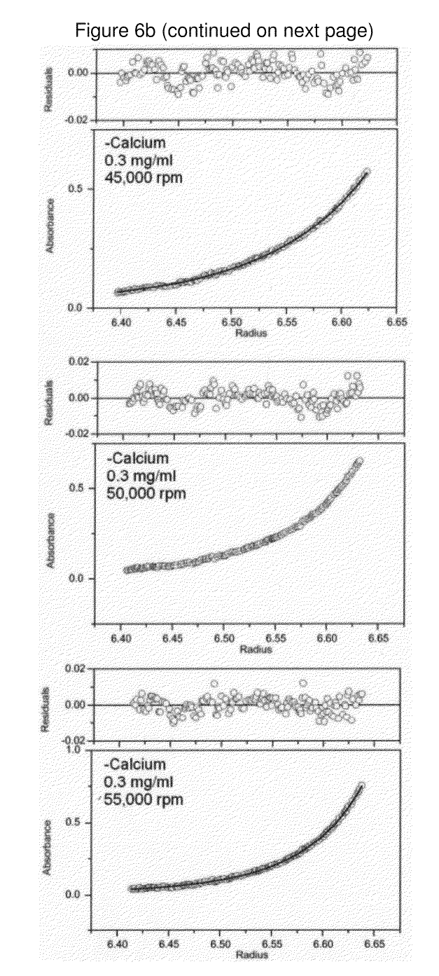

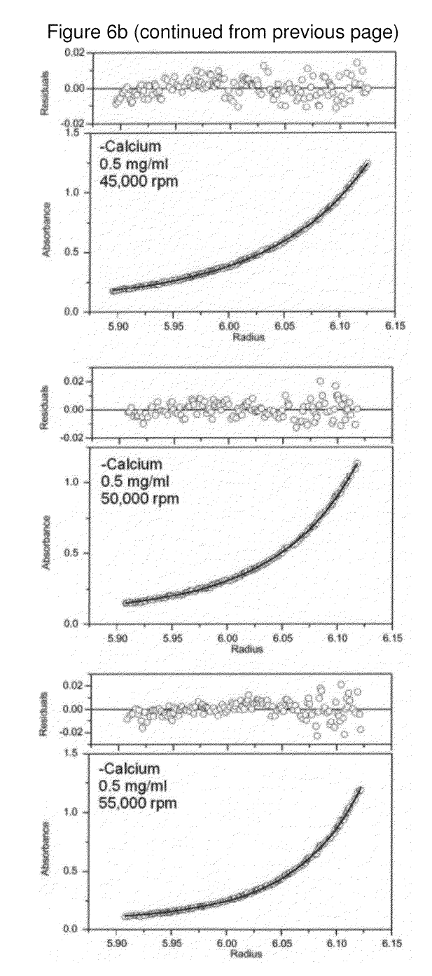

The hydroxyapatite lattice binding mode represented by the best solution presupposes that osteocalcin engages hydroxyapatite with the acidic Ca²⁺-coordinating surface as a monomer. In the crystal structure of porcine osteocalcin, however, the five Ca²⁺ ions are sandwiched between two crystallographically related protein molecules. If overly stable, this dimeric state could present an impediment to hydroxyapatite binding. To investigate whether porcine osteocalcin exists as a monomer or dimer in solution, sedimentation equilibrium analysis in the presence and absence of 10 mM CaCl₂ was carried out. In both cases, the sedimentation equilibrium data were best fitted to a monomer-dimer equilibrium model (FIG. 6). The extrapolated dissociation constants (K_{d}) for the osteocalcin dimer were 8×10⁻⁴ M and 2×10⁻⁴M in the absence and presence of 10 mM CaCl₂, respectively. Theses K_{d} values are 2-3 orders of magnitude higher than the concentration of osteocalcin in human serum (0.9×10⁻⁶ to 7×10⁻¹⁶ M),³¹ showing that osteocalcin exists as a monomer in vivo.

The crystal structure of porcine osteocalcin provides a first glimpse of the underlying interactions that may constitute biomineral recognition. The recognition of crystal lattices by proteins is important in many biological processes, including the inhibition of ice crystal growth and the development of teeth, bone and shells. The best-characterized protein-crystal recognition system studied so far corresponds to the interaction of antifreeze proteins (AFP) with ice.^{32, 33} AFPs bind to the surface of ice to modify crystal morphology and to inhibit ice growth. AFPs with different three-dimensional structures bind to different planes of ice, and the shape complementarity between the ice-binding surface of AFP and the ice crystal surface to which it binds is the primary determinant for binding specificity.

The excellent surface complementarity between the Ca²⁺-coordinating surface of porcine osteocalcin and the prism face of hydroxyapatite shows that porcine osteocalcin will also show selective binding characteristics to hydroxyapatite. By analogy to the antifreeze proteins, the binding of osteocalcin to hydroxyapatite could directly modulate hydroxyapatite crystal morphology and growth. In addition, when osteocalcin is bound to the surface of hydroxyapatite, other regions, including the carboxy terminus of the protein, which possesses chemotactic activity,²⁴ would be well orientated to carry out recruitment and signal transduction functions through binding to cell surface receptors on osteoclasts²³ and osteoblasts.²²

### Three-Dimensional Configurations

X-ray structure coordinates define a unique configuration of points in space. Those of skill in the art understand that a set of structure coordinates for protein or a protein/ligand complex, or a portion thereof, define a relative set of points that, in turn, define a configuration in three dimensions. A similar or identical configuration can be defined by an entirely different set of coordinates, provided the relative distances and angles between coordinates remain essentially the same.

The configurations of points in space derived from structure coordinates according to the invention can be visualized as, for example, a holographic image, a stereodiagram, a model or a computer-displayed image, and the invention thus includes such images, diagrams or models.

### Structurally Equivalent Crystal Structures

Various computational analyses can be used to determine whether a molecule or the active site portion thereof is “structurally equivalent,” defined in terms of its three-dimensional structure, to all or part of osteocalcin or its binding sites. Such analyses may be carried out in current software applications, such as the Molecular Similarity application of QUANTA (Molecular Simulations Inc., San Diego, Calif.) version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: (1) load the structures to be compared; (2) define the atom equivalences in these structures; (3) perform a fitting operation; and (4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention equivalent atoms are defined as protein backbone atoms (N, C.alpha, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or active site thereof, or any portion thereof, that has a root mean square deviation of conserved residue backbone atoms (N, Cα, C, O) of less than about 1.5 Å, when superimposed on the relevant backbone atoms described by the reference structure coordinates listed in the tables, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structure coordinates listed in the tables, plus/minus a root mean square deviation from the conserved backbone atoms of those amino acids of not more than 1.5 Å. More preferably, the root mean square deviation is less than about 1.0 Å or 0.5 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of osteocalcin or a binding site portion thereof, as defined by the structure coordinates of osteocalcin described herein.

### Structurally Homologous Molecules, Molecular Complexes, and Crystal Structures

The structure coordinates are useful to obtain structural information about another crystallized molecule or molecular complex. The method of the invention allows determination of at least a portion of the three-dimensional structure of molecules or molecular complexes which contain one or more structural features that are similar to structural features of osteocalcin. These molecules are referred to herein as “structurally homologous” to osteocalcin. Similar structural features can include, for example, regions of amino acid identity, conserved active site or binding site motifs, and similarly arranged secondary structural elements (e.g., α-helices and β-sheets).Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by,³⁴ and available from the National Center for Biotechnology Information (NCBI of the U.S. National Institutes of Health. Preferably, the default values for all BLAST 2 search parameters are used. In the comparison of two amino acid sequences using the BLAST search algorithm, structural similarity is referred to as “identity.” Preferably, a structurally homologous molecule is a protein that has an amino acid sequence sharing at least 50%, 60%, 70%, 75%, 80%, 85%, 90%, 95%, 96%, 97%, 98% or 99% sequence identity with a native or recombinant amino acid sequence of osteocalcin. More preferably, a protein that is structurally homologous to osteocalcin includes at least one contiguous stretch of at least 25 or 50 amino acids that shares at least 80% amino acid sequence identity with the analogous portion of the native or recombinant osteocalcin. Methods for generating structural information about the structurally homologous molecule or molecular complex are well-known and include, for example, molecular replacement techniques.

Therefore, in another embodiment this invention provides a method of utilizing molecular replacement to obtain structural information about a molecule or molecular complex whose structure is unknown comprising the steps of:

(a) crystallizing the molecule or molecular complex of unknown structure;
(b) generating an x-ray diffraction pattern from said crystallized molecule or molecular complex; and
(c) applying at least a portion of the structure coordinates to the x-ray diffraction pattern to generate a three-dimensional electron density map of the molecule or molecular complex whose structure is unknown.

By using molecular replacement, all or part of the structure coordinates of osteocalcin as provided by this invention can be used to determine the structure of a crystallized molecule whose structure is unknown more quickly and efficiently than attempting to determine such information ab initio.

Molecular replacement provides an accurate estimation of the phases for an unknown structure. Phases are a factor in equations used to solve crystal structures that cannot be determined directly experimentally. Obtaining accurate values for the phases, by methods other than molecular replacement, is a time-consuming process that involves iterative cycles of approximations and refinements and greatly hinders the solution of crystal structures. However, when the crystal structure of a protein containing at least a structurally homologous portion has been solved, the phases from the known structure provide a satisfactory estimate of the phases for the unknown structure.

Thus, this method involves generating a preliminary model of a molecule whose structure coordinates are unknown, by orienting and positioning the relevant portion of osteocalcin to the structure coordinates listed within the unit cell of the crystal of the unknown molecule or molecular complex so as best to account for the observed x-ray diffraction pattern of the crystal of the molecule whose structure is unknown. Phases can then be calculated from this model and combined with the observed x-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex.^{35, 36}

Structural information about a portion of any crystallized molecule that is sufficiently structurally homologous to a portion of osteocalcin can be resolved by this method. In addition to a molecule that shares one or more structural features with osteocalcin as described above, a molecule that has similar bioactivity, such as the same hydroxapatite binding activity as osteocalcin, may also be sufficiently structurally homologous to osteocalcin to permit use of the structure coordinates of osteocalcin to solve its crystal structure.

In a preferred embodiment, the method of molecular replacement is utilized to obtain structural information about a molecule, wherein the molecule comprises at least one osteocalcin fragment or homolog. A “fragment” of osteocalcin is an osteocalcin molecule that has been truncated at the N-terminus or the C-terminus, or both. In the context of the present invention, a “homolog” of osteocalcin is a protein that contains one or more amino acid substitutions, deletions, additions, or rearrangements with respect to the amino acid sequence of osteocalcin, but that, when folded into its native conformation, exhibits or is reasonably expected to exhibit at least a portion of the tertiary (three-dimensional) structure of osteocalcin. For example, structurally homologous molecules can contain deletions or additions of one or more contiguous or noncontiguous amino acids, such as a loop or a domain. Structurally homologous molecules also include “modified” osteocalcin molecules that have been chemically or enzymatically derivatized at one or more constituent amino acids, including side chain modifications, backbone modifications, and N- and C-terminal modifications including acetylation, hydroxylation, methylation, amidation, and the attachment of carbohydrate or lipid moieties, cofactors, and the like.

A heavy atom derivative of osteocalcin is also included as an osteocalcin. The term “heavy atom derivative” refers to derivatives of osteocalcin produced by chemically modifying a crystal of osteocalcin. In practice, a crystal is soaked in a solution containing heavy metal atom salts, or organometallic compounds, e.g., lead chloride, gold thiomalate, thiomersal or uranyl acetate, which can diffuse into the crystal and bind to the surface of the protein. The locations of the bound heavy metal atoms can be determined by x-ray diffraction analysis of the soaked crystal. This information, in turn, is used to generate the phase information used to construct three-dimensional structure of the protein.³⁷

The structure coordinates of osteocalcin as provided by this invention are particularly useful in solving the structure of osteocalcin mutants. Mutants are prepared, for example, by expression of osteocalcin cDNA previously altered in its coding sequence by oligonucleotide-directed mutagenesis. Mutants are generated by site-specific incorporation of unnatural amino acids into osteocalcin proteins using the general biosynthetic method of.³⁸ In this method, the codon encoding the amino acid of interest in wild-type osteocalcin is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant with the site-specific incorporated unnatural amino acid.

The structure coordinates of osteocalcin are also particularly useful to solve the crystal structure of osteocalcin mutants. This approach enables the determination of the optimal sites for interaction, including candidate osteocalcin inhibitors/modulators. Potential sites for modification within the various binding sites of the molecule can also be identified. This information provides an additional tool for determining the most efficient binding interactions. For example, high resolution x-ray diffraction data collected from crystals exposed to different types of solvent allows the determination of where each type of solvent molecule resides. Small molecules that bind tightly to those sites can then be designed and synthesized and tested for their osteocalcin inhibition activity.

All of the complexes referred to above may be studied using well-known x-ray diffraction techniques and may be refined versus 1.5-3.0 Å resolution x-ray data to an R value of about 0.20 or less using computer software, such as CNS. This information may thus be used to optimize known osteocalcin inhibitors/modulators, and more importantly, to design new osteocalcin inhibitors/modulators.

The invention also includes the unique three-dimensional configuration defined by a set of points defined by the structure coordinates for a molecule structurally homologous to osteocalcin as determined using the method of the present invention, structurally equivalent configurations, and magnetic storage media comprising such set of structure coordinates.

Further, the invention includes structurally homologous molecules as identified using the method of the invention.

### Homology Modeling

Using homology modeling, a computer model of an osteocalcin homolog can be built or refined without crystallizing the homolog. First, a preliminary model of the osteocalcin homolog is created by sequence alignment with osteocalcin, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. Where the osteocalcin homolog has been crystallized, the final homology model can be used to solve the crystal structure of the homolog by molecular replacement, as described above. Next, the preliminary model is subjected to energy minimization to yield an energy minimized model. The energy minimized model may contain regions where stereochemistry restraints are violated, in which case such regions are remodeled to obtain a final homology model.

### Drug Design of Inhibitors

### Inhibitors

Inhibitors of osteocalcin provide a basis for diagnosis and/or treatment of bone-related pathologies. “Pathology” includes a disease, a disorder and/or an abnormal physical state caused by increased or decreased bone resorption/formation and bony metastases of cancers such as bone metabolic disorders, osteoporosis, breast cancer, prostate cancer, lung cancer and hypercalcemia malignancy. The structures are useful in the design of inhibitors, which may be used as therapeutic or prophylactic compounds for treating pathologies in which downregulation of osteocalcin-hydroxapatite binding is beneficial. It will be apparent that methods using osteocalcin described below may be readily adapted for use with a fragment of osteocalcin or osteocalcin variant.

The characterization of the novel binding surface permits the design of potent, highly selective inhibitors. Several approaches can be taken for the use of the structure in the rational design of inhibitors. A computer-assisted, manual examination of an inhibitor binding site structure may be done.

### Rational Drug Design

Computational techniques can be used to screen, identify, select and design chemical entities capable of associating with osteocalcin or structurally homologous molecules. Knowledge of the structure coordinates for osteocalcin permits the design and/or identification of synthetic compounds and/or other molecules which have a shape complementary to the conformation of an osteocalcin binding site. In particular, computational techniques can be used to identify or design chemical entities, such as inhibitors, agonists and antagonists, that associate with an osteocalcin binding site. Inhibitors may bind to or interfere with all or a portion of the binding site of osteocalcin to hydroxyapatite, and can be competitive, non-competitive, or uncompetitive inhibitors. Once identified and screened for biological activity, these inhibitors/agonists/antagonists may be used therapeutically or prophylactically to block osteocalcin activity. Structure-activity data for analogs of ligands that bind to or interfere with osteocalcin-like binding sites can also be obtained computationally.

Accordingly, the invention includes a method of designing a compound that inhibits osteocalcin activity, comprising performing rational drug design with a 3D structure of osteocalcin or fragment thereof to design a compound that interacts with the 3D structure of hydroxyapatite or fragment thereof and inhibits osteocalcin activity.

The invention also includes a method of identifying whether a compound inhibits osteocalcin activity, comprising performing rational drug design with a 3D structure of osteocalcin or fragment thereof, the drug design comprising i) comparing the 3D structure of the compound to the 3D structure of osteocalcin or fragment thereof and ii) determining whether the compound interacts with the 3D structure of hydroxyapatite and inhibits osteocalcin activity.

The drug design is preferably performed in conjunction with computer modeling comprising introducing into a computer program structural coordinates defining an osteocalcin or fragment thereof, wherein the program generates the 3D structure of the osteocalcin or fragment.

In the method, the compound that inhibits osteocalcin preferably has a greater affinity for the binding of hydroxyapatite than does osteocalcin.

The term “osteocalcin-like binding site” refers to a portion of a molecule or molecular complex whose shape is sufficiently similar to at least a portion of the active site of osteocalcin as to be expected to bind hydroxyapatite. A structurally equivalent active site is defined by a root mean square deviation from the structure coordinates of the backbone atoms of the amino acids that make up the active site in osteocalcin of at most about 1.5 Å. How this calculation is obtained is described below.

Accordingly, the invention thus provides molecules or molecular complexes comprising an osteocalcin binding site or an osteocalcin-like binding site, as defined by the sets of structure coordinates described above.

The term “chemical entity,” as used herein, refers to chemical compounds, complexes of two or more chemical compounds, and fragments of such compounds or complexes. Chemical entities that are determined to associate with osteocalcin are potential drug candidates.

Data stored in a machine-readable storage medium that is capable of displaying a graphical three-dimensional representation of the structure of osteocalcin or a structurally homologous molecule, as identified herein, or portions thereof may thus be advantageously used for drug discovery. The structure coordinates of the chemical entity are used to generate a three-dimensional image that can be computationally fit to the three-dimensional image of osteocalcin or a structurally homologous molecule. The three-dimensional molecular structure encoded by the data in the data storage medium can then be computationally evaluated for its ability to associate with hydroxyapatite. When the molecular structures encoded by the data are displayed in a graphical three-dimensional representation on a computer screen, the protein structure can also be visually inspected for potential association with hydroxyapatite.

One embodiment of the method of drug design involves evaluating the potential association of a known chemical entity with osteocalcin, a structurally homologous molecule or with the binding-site of osteocalcin on hydroxyapatite. The method of drug design thus includes computationally evaluating the potential of a selected chemical entity to associate with any of the molecules or molecular complexes set forth above. This method comprises the steps of: (a) employing computational means to perform a fitting operation between the selected chemical entity and a binding site, or a pocket nearby the substrate binding site, of the molecule or molecular complex; and (b) analyzing the results of said fitting operation to quantify the association between the chemical entity and the active site.

In another embodiment, the method of drug design involves computer-assisted design of chemical entities that associate with osteocalcin, its homologs, portions thereof, or with the binding site of osteocalcin on hydroxyapatite. Chemical entities can be designed in a step-wise fashion, one fragment at a time, or may be designed as a whole or “de novo.”

To be a viable drug candidate, the chemical entity identified or designed according to the method must be capable of structurally associating with at least part of osteocalcin or osteocalcin binding sites on hydroxyapatite, and must be able, sterically and energetically, to assume a conformation that allows it to associate. Non-covalent molecular interactions important in this association include hydrogen bonding, van der Waals interactions, hydrophobic interactions, and electrostatic interactions. Conformational considerations include the overall three-dimensional structure and orientation of the chemical entity in relation to the active site, and the spacing between various functional groups of an entity that directly interact with the osteocalcin-like active site or homologs thereof.

Optionally, the potential binding of a chemical entity to an osteocalcin or osteocalcin binding site on hydroxyapatite is analyzed using computer modeling techniques prior to the actual synthesis and testing of the chemical entity. If these computational experiments suggest insufficient interaction and association, testing of the entity is obviated. However, if computer modeling indicates a strong interaction, the molecule may then be synthesized and tested for its ability to interfere with an osteocalcin binding to hydroxyapatite. Binding assays to determine if a compound actually binds can also be performed and are well known in the art. Binding assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

One skilled in the art may use one of several methods to screen chemical entities or fragments for their ability to associate with an osteocalcin or osteocalcin binding site on hydroxyapatite. This process may begin by visual inspection on the computer screen based on the osteocalcin structure coordinates or other coordinates which define a similar shape generated from the machine-readable storage medium. Selected fragments or chemical entities may then be positioned in a variety of orientations, or docked, within the active site. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical entities. Examples include GRID³⁹ (available from Oxford University, Oxford, UK); MCSS⁴⁰ (available from Molecular Simulations, San Diego, Calif.); AUTODOCK⁴¹ (available from Scripps Research Institute, La Jolla, Calif.); and DOCK⁴² (available from University of California, San Francisco, Calif.).

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of osteocalcin. This could be followed by manual model building using software such as QUANTA or SYBYL (Tripos Associates, St. Louis, Mo.).

Useful programs to aid one of skill in the art in connecting the individual chemical entities or fragments include, without limitation, CAVEAT^{43, 44} (available from the University of California, Berkeley, Calif.); 3D database systems such as ISIS (available from MDL Information Systems, San Leandro, Calif.) reviewed in Y. C. Martin⁴⁵; and HOOK⁴⁶ (available from Molecular Simulations, San Diego, Calif.).

Osteocalcin or hydroxyapatite binding compounds may be designed “de novo” using either an empty binding site or optionally including some portion(s) of a known inhibitor(s). There are many de novo ligand design methods including, without limitation, LUDI⁴⁷ (available from Molecular Simulations Inc., San Diego, Calif.); LEGEND⁴⁸ (available from Molecular Simulations Inc., San Diego, Calif.); LeapFrog (available from Tripos Associates, St. Louis, Mo.); and SPROUT⁴⁹ (available from the University of Leeds, UK).

Once a compound has been designed or selected by the above methods, the efficiency with which that entity may bind to or interfere with an osteocalcin or osteocalcin binding site on hydroxyapatite may be tested and optimized by computational evaluation.

An entity designed or selected as binding to or interfering with an osteocalcin may be further computationally optimized so that in its bound state it would preferably lack repulsive electrostatic interaction with its target and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. 15106); AMBER, version 4.1 (P. A. Kollman, University of California at San Francisco, 94143); QUANTA/CHARMM (Molecular Simulations, Inc., San Diego, Calif. 92121); Insight II/Discover (Molecular Simulations, Inc., San Diego, Calif. 92121); DelPhi (Molecular Simulations, Inc., San Diego, Calif. 92121); and AMSOL (Quantum Chemistry Program Exchange, Indiana University). These programs may be implemented, for instance, using a Silicon Graphics workstation such as an Indigo.sup.2 with “IMPACT” graphics. Other hardware systems and software packages will be known to those skilled in the art.

Another approach encompassed by this invention is the computational screening of small molecule databases for chemical entities or compounds that can bind in whole, or in part, to an osteocalcin or osteocalcin binding site on hydroxyapatite. In this screening, the quality of fit of such entities to the binding site may be judged either by shape complementarity or by estimated interaction energy.⁵⁰

Yet another approach to rational drug design involves probing the osteocalcin crystal of the invention with molecules comprising a variety of different functional groups to determine optimal sites for interaction between candidate inhibitors and the protein. For example, high resolution x-ray diffraction data collected from crystals soaked in or co-crystallized with other molecules allows the determination of where each type of solvent molecule sticks. Molecules that bind tightly to those sites can then be further modified and synthesized and tested for their osteocalcin-hydroxyapatite inhibitor/modulator activity.⁵¹

In a related approach, iterative drug design is used to identify inhibitors of osteocalcin. Iterative drug design is a method for optimizing associations between a protein and a compound by determining and evaluating the three-dimensional structures of successive sets of protein/compound complexes. In iterative drug design, crystals of a series of protein/compound complexes are obtained and then the three-dimensional structures of each complex are solved. Such an approach provides insight into the association between the proteins and compounds of each complex. This is accomplished by selecting compounds with inhibitory activity, obtaining crystals of this new protein/compound complex, solving the three dimensional structure of the complex, and comparing the associations between the new protein/compound complex and previously solved protein/compound complexes. By observing how changes in the compound affected the protein/compound associations, these associations may be optimized.

A compound that is identified or designed as a result of any of these methods can be obtained (or synthesized) and tested for its biological activity, e.g., modulation of osteocalcin-hydroxyapatite binding. Patents relating to drug design and other methods described herein include U.S. Pat. Nos. 6,801,860, 6,794,146, 6,451,575 6,303,287, 6,083,711, 6,274,336, 6,266,622 and 6,197,495 which are incorporated by reference herein in their entirety.

### Apparatus Including the Osteocalcin 3D Structure or Other Osteocalcin Structural Information

Storage media for the osteocalcin 3D structure or other osteocalcin structural information include, but are not limited to: magnetic storage media, such as floppy discs; hard disc storage medium, and magnetic tape; optical storage media such as optical discs or CD-ROM; electrical storage media such as RAM and ROM; and hybrids of these categories such as magnetic/optical storage media. Any suitable computer readable mediums can be used to create a manufacture comprising a computer readable medium having recorded on it an amino acid sequence and/or data of the present invention.

“Recorded” refers to a process for storing information on computer readable medium. A skilled artisan can readily adopt any of the presently known methods for recording information on computer readable medium to store an amino acid sequence, nucleotide sequence and/or EM data information of the present invention.

A variety of data storage structures are available to a skilled artisan for creating a computer readable medium having recorded thereon an amino acid sequence and/or data of the present invention. The choice of the data storage structure will generally be based on the means chosen to access the stored information. In addition, a variety of data processor programs and formats can be used to store the sequence and data information of the present invention on computer readable medium. The sequence information can be represented in a word processing text file, formatted in commercially-available software such as WordPerfect and MicroSoft Word, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the information of the present invention.

By providing the sequence and/or data on computer readable medium and the structural information in this application, a skilled artisan can routinely access the sequence and data to model an osteocalcin, a subdomain thereof, or a ligand thereof. As described above, computer algorithms are publicly and commercially available which allow a skilled artisan to access this data provided in a computer readable medium and analyze it for molecular modeling or other uses.

The present invention further provides systems, particularly computer-based systems, which contain the sequence and/or data described herein. Such systems are designed to do molecular modeling for an osteocalcin or at least one subdomain or fragment thereof.

In one embodiment, the system includes a means for producing a 3D structure of osteocalcin (or a fragment or derivative thereof) and means for displaying the 3D structure of osteocalcin. The system is capable of carrying out the methods described in this application. The system preferably further includes a means for comparing the structural coordinates of a candidate compound to the structural coordinates of the osteocalcin (or a fragment or derivative thereof, such as an active site or other region described in this application) and means for determining if the candidate compound is capable of modulating osteocalcin, as described in the methods of the invention.

As used herein, “a computer-based system” refers to the hardware means, software means, and data storage means used to analyze the sequence and/or data of the present invention. The minimum hardware means of the computer-based systems of the present invention comprises a central processing unit (CPU), input means, output means, and data storage means. A skilled artisan can readily appreciate which of the currently available computer-based systems are suitable for use in the present invention.

As stated above, the computer-based systems of the present invention comprise a data storage means having stored therein an osteocalcin or fragment sequence and/or data of the present invention and the necessary hardware means and software means for supporting and implementing an analysis means. As used herein, “data storage means” refers to memory which can store sequence or data (coordinates, distances, 3D structure etc.) of the present invention, or a memory access means which can access manufactures having recorded thereon the sequence or data of the present invention.

As used herein, “search means” or “analysis means” refers to one or more programs which are implemented on the computer-based system to compare a target sequence or target structural motif with the sequence or data stored within the data storage means. Search means are used to identify fragments or regions of an osteocalcin which match a particular target sequence or target motif. A variety of known algorithms are disclosed publicly and a variety of commercially available software for conducting search means are and can be used in the computer-based systems of the present invention. A skilled artisan can readily recognize that any one of the available algorithms or implementing software packages for conducting computer analyses can be adapted for use in the present computer-based systems.

As used herein, “a target structural motif,” or “target motif,” refers to any rationally selected sequence or combination of sequences in which the sequences(s) are chosen based on a three-dimensional configuration or electron density map which is formed upon the folding of the target motif. There are a variety of target motifs known in the art. Protein targets include, but are not limited to, active sites, structural subdomains, epitopes, and functional domains. A variety of structural formats for the input and output means can be used to input and output the information in the computer-based systems of the present invention.

One application of this embodiment provides a block diagram of a computer system that can be used to implement the present invention. The computer system includes a processor connected to a bus. Also connected to the bus are a main memory (preferably implemented as random access memory, RAM) and a variety of secondary storage memory such as a hard drive and a removable storage medium. The removable medium storage device may represent, for example, a floppy disk drive, A CD-ROM drive, a magnetic tape drive, etc. A removable storage unit (such as a floppy disk, a compact disk, a magnetic tape, etc.) containing control logic and/or data recorded therein may be inserted into the removable medium storage medium. The computer system includes appropriate software for reading the control logic and/or the data from the removable medium storage device once inserted in the removable medium storage device. A monitor can be used as connected to the bus to visualize the structure determination data.

Amino acid, encoding nucleotide or other sequence and/or data of the present invention may be stored in a well known manner in the main memory, any of the secondary storage devices, and/or a removable storage device. Software for accessing and processing the amino acid sequence and/or data (such as search tools, comparing tools, etc.) reside in main memory during execution.

One or more computer modeling steps and/or computer algorithms are used as described above to provide a molecular 3-D model, preferably showing the 3-D structure, of a cleaved osteocalcin, using amino acid sequence data and atomic coordinates for the osteocalcin. The structure of other osteocalcin-like molecules may be readily determined using methods of the invention and the present knowledge of these molecules.

Accordingly, the invention provides computer media and systems for performing a method of the invention. The invention includes a computer readable media, such as a disk (eg. hard disk, floppy disk, CD-ROM, CD-RW, DVD), with structural coordinate data of Table 3, recorded thereon, osteocalcin bound to an inhibitor, substrate or a fragment of the foregoing recorded thereon, the structure data being derivable from the structural coordinate data of Table 3. The structural coordinate data is optionally obtained by x-ray diffraction with a crystal of the invention.

Another aspect of the invention relates to a computer system, intended to generate structures and/or perform rational drug design for osteocalcin, the system containing structural coordinate data of Table 3, said data defining the 3D structure of osteocalcin, osteocalcin bound to an inhibitor, substrate or a fragment of the foregoing, said structure data being derivable from the atomic coordinate data of Table 3. The structural coordinate data is optionally obtained by x-ray diffraction with a crystal of the invention.

### Osteocalcin Derivatives

The invention also includes the use of osteocalcin derivatives to interfere with osteocalcin binding to hydroxyapatite. Such derivatives include those that retain the features on the hydroxyapatite binding surface but change the features on the remaining surfaces such that osteocalcin will not interact with cells/proteins in the original manner. Examples include:

(i) osteocalcin purified from non-human species, including but not limiting to pig, monkey, cow, sheep, goat, dog, cat, rabbit, wallaby, rat, mouse, xenopus, emu, chicken, carp, tetraodon, fugu, bluegill, seabream, swordfish, other fish species, bird species, non-vertebrates
ii) mutating residues in 1-16 and/or 25-end;
iii) insertion of residues into 1-16 and/or 25-end;
iv) deletion of residues from 1-16 and/or 25-end;
v) chemical modification of residues in 1-16 and/or 25-end using standing chemical modification techniques.
(vi) crosslinking of osteocalcin to other proteins, peptides or structures.

In another embodiment, derivatives are made such that only the features on the hydroxyapatite binding surface are altered. This will direct the osteocalcin derivatives to locations other than bone and allow it to compete with the bone-bound osteocalcin in interacting with cellular protein and reduce the recruitment of cell to bone. Such osteocalcin derivatives may include, but are not limited to

i) de-carboxylation of Glas by chemical means or by expressing osteocalcin in host cells that cannot synthesize GLA.
ii) Mutation of residues on the surface, as can be deduced from the 3-D structure (including but not limiting to Gla-17, Gla-21, Gla-24, Asp-30, Asp-34), that can cause steric clash with the hydroxyapatite surface and therefore prevent the OC mutant from interacting with the HA surface.

In a further embodiment, bisphosphonate derivatives and tetracycline derivatives may be used to bind the hydroxyapatite surface, thereby inhibiting osteocalcin binding. Bisphosphonate and tetracycline derivatives have been effective in treating osteoporosis, breast cancer and/or prostate cancer. The main site of action of bisphosphonates and tetracycline derivative is likely related to its ability to bind to hydroxyapatite. The exposed surface on hydroxyapatite is likely mainly planar and therefore the interaction of bisphosphonate and tetracycline will involve the planar surface on these molecules. The planar surface has a lot of electronegative moieties that can presumably interact with Ca and therefore bind to hydroxyapatite. Compounds or derivatives of tetracycline and bisphosphonates can be designed with an aim to:

(i) increase the planar surface area;
(ii) optimize its interaction with hydroxyapatite surface;
(iii) optimize its van der Waals interaction with the hydroxyapatite surface;
(iv) optimize its electrostatic interaction with the hydroxyapatite surface;
(v) optimize its bioavailability;
(vi) optimize its biostability; and/or
(vii) optimize its absorption through the digestive system.

Examples of bisphosphonates that can be used or modified, include but are not limited to, pyrophosphate, bisphosphonate, etidronate, clodronate, pamidronate, tiludronate, risedronate, zoledronate, alendronate, YM-175, and ibandronate. The two phosphates on all bisphosphonates are located on one side of the molecule. The design of a new compound would therefore involve addition of phosphates or other electronegative moieties to the carbon linking the two phosphates or to any other atoms on the structure such that the newly added phosphate or electronegative moiety will be positioned on the same plane as the two existing phosphates.

The design of compounds may achieve at least one of the following properties:

(i) bind to hydroxyapatite tighter than osteocalcin
(ii) have less toxicity
(iii) displace osteocalcin more efficiently.
Assays of Osteocalcin or Other Derivatives or Inhibitors Identified from the Osteocalcin Structure

Once identified, the inhibitor may then be tested for bioactivity using standard techniques (e.g. in vitro or in vivo assays). For example, the compound identified by drug design may be used in binding assays using conventional formats to screen agonists/antagonists (e.g by measuring in vivo or in vitro binding of osteocalcin after addition of a compound). Suitable assays include, but are not limited to, the enzyme-linked immunosorbent assay (ELISA), or a fluorescence quench assay. In evaluating osteocalcin modulators for biological activity in animal models (e.g. rat, mouse, rabbit), various oral and parenteral routes of administration are evaluated.

The method may also comprise obtaining or synthesizing the compound and determining whether the compound modulates the activity of the osteocalcin, fragment or derivative in an in vivo or in vitro assay. Such an assay optionally comprises:

a) obtaining osteocalcin with recombinant technology, chemical synthesis or purification from bone;
b) contacting osteocalcin with hydroxyapatite;
c) adding a test compound to compete with the bound osteocalcin for hydroxyapatite; and
d) measuring the amount of osteocalcin dissociated from hydroxyapatite as a result of the addition of the test compound;
whereby a compound that competes with osteocalcin for hydroxyapatite is identified as a compound that inhibits osteocalcin activity.

Preferably, inhibitors may be used in a screening assay involving the following steps:

(i) Label osteocalcin with fluorescence;
(ii) incubate labeled osteocalcin with hydroxyapatite powder;
(iii) wash off excess osteocalcin;
(iv) add different concentrations of the compound to be analysed;
(v) measure the release of osteocalcin from the hydroxyapatite-osteocalcin complex by an appropriate spectrophotometer, for example, fluorescence spectrophotometer, fluorescence polarization spectrophotometer); and
(vi) determine the potency of the compound in releasing osteocalcin.

Furthermore, this screening assay may be carried out in a high throughput manner using a robotic system.

In summary, the methods of the invention optionally involve providing a test (candidate) compound, identifying whether the compound interacts (eg. fits spatially) with one or more atoms described herein that are useful for drug design methods and assaying the ability of the compound to modulate osteocalcin activity.

### Pharmaceutical/Diagnostic Formulations, Methods of Medical Treatment and Uses

Medical Treatments and Uses

Abnormal bone turnover causes many diseases in mammals, such as humans. Examples of these diseases include: bone metabolic disorders, osteoporosis, breast cancer, prostate cancer, lung cancer and hypercalcemia malignancy.

Accordingly, the invention includes a method of medical prevention or treatment of a disease, preferably bone disease, in a subject having bone metabolic disorders, osteoporosis, breast cancer, prostate cancer, lung cancer and hypercalcemia, comprising administering to the subject a compound or derivative of the invention or a compound described in this application and/or identified by a method of the invention. The invention also includes the use of such compounds/derivatives for prevention or treatment of a disease, preferably bone disease, in a subject having bone metabolic disorders, osteoporosis, breast cancer, prostate cancer, lung cancer and hypercalcemia. The invention also includes the use of the compounds/derivatives for preparation of a medicament (pharmaceutical substance), for example, for prevention or treatment of the aforementioned diseases and disorders.

The term “effective amount” means an amount effective, at dosages and for periods of time necessary to treat bone disease or disorder.

The term “administering” is defined as any conventional route for administering a drug as is known to one skilled in the art. This may include, for example, administration via the oral, parenteral (i.e. subcutaneous, intradermal, intramuscular, etc.) or mucosal surface route. One skilled in the art will appreciate that the dosage regime can be determined and/or optimized without undue experimentation.

### Pharmaceutical Compositions

Inhibitors may be combined in pharmaceutical compositions according to known techniques. The compounds/derivatives are preferably incorporated into pharmaceutical dosage forms suitable for the desired administration route such as tablets, dragees, capsules, granules, suppositories, solutions, suspensions and lyophilized compositions to be diluted to obtain injectable liquids. The dosage forms are prepared by conventional techniques and in addition to the inhibitor could contain solid or liquid inert diluents and carriers and pharmaceutically useful additives such as lipid vesicles liposomes, aggregants, disaggregants, salts for regulating the osmotic pressure, buffers, sweeteners and colouring agents. Slow release pharmaceutical forms for oral use may be prepared according to conventional techniques. Other pharmaceutical formulations are described for example in U.S. Pat. No. 5,192,746.

Pharmaceutical compositions used to treat patients having diseases, disorders or abnormal physical states could include a compound of the invention and an acceptable vehicle or excipient [61 and subsequent editions]. Vehicles include saline and D5W (5% dextrose and water). Excipients include additives such as a buffer, solubilizer, suspending agent, emulsifying agent, viscosity controlling agent, flavor, lactose filler, antioxidant, preservative or dye. The compound may be formulated in solid or semisolid form, for example pills, tablets, creams, ointments, powders, emulsions, gelatin capsules, capsules, suppositories, gels or membranes. Routes of administration include oral, topical, rectal, parenteral (injectable), local, inhalant and epidural administration. The compositions of the invention may also be conjugated to transport molecules to facilitate transport of the molecules. The methods for the preparation of pharmaceutically acceptable compositions which can be administered to patients are known in the art.

The pharmaceutical compositions can be administered to humans or animals. Dosages to be administered depend on individual patient condition, indication of the drug, physical and chemical stability of the drug, toxicity, the desired effect and on the chosen route of administration.⁵²

The present invention has been described in detail and with particular reference to the preferred embodiments; however, it will be understood by one having ordinary skill in the art that changes can be made thereto without departing from the spirit and scope thereof.

### Methods

### Protein Production and Purification

Osteocalcin was extracted from its natural source, bone, and purification was carried out based on the previously described protocol⁵³ with modification for scale-up production. The diaphysis of femur bone was separated from the epiphysis with a band saw and flesh was removed from the diaphysis with a razor blade and wood scraper. After soaking in cold acetone for 10 minutes, the periosteum lining was removed with a wood scraper and steel wool. The marrow was subsequently removed with a long spatula and the medullary cavity was cleaned with a test tube brush in warm soap water. The cleaned bone diaphysis was cut longitudinally in half then crosscut into thin slices (˜2 mm) then frozen in liquid nitrogen and lyophilized overnight (FTS Systems Inc.). The lyophilized bone was frozen in liquid nitrogen then ground into powder with a stainless steel blender (Waring Commercial Blender) followed by a coffee grinder (Braun). The bone powder was sieved through a stainless steel mesh yielding bone powder with average size of 200 μm. The fine powder (30 grams) was washed two times with 500 ml of cold water containing 0.6 mM PMSF at 4° C. for 30 minutes and the pellet was collected after centrifugation (Sorvall) at 1500×g for 30 minutes. The pellet was frozen in liquid nitrogen and lyophilized. The freeze-dried powder was demineralized at 4° C. by gentle stirring for 4 hours in 300 ml of 20% formic acid (HCOOH) containing 0.6 mM PMSF. The solution was then centrifuged at 40,000×g for 60 minutes. The insoluble pellet was resuspended with 50 ml of 20% formic acid, stirred for 30 minutes to release any trapped proteins, and recentrifuged. The 40,000×g supernatants were combined and filtered (Millipore AP2004700).

The filtered supernatant was made to 0.1% Trifluoroacetic Acid (CF₃COOH) (TFA). Sep-Pak C18 cartridges (Waters No. WAT043345) were mounted onto a 1-liter flask under vacuum, conditioned with sequential addition of 100 ml Methanol (CH₃OH) and 100 ml of 0.1% TFA, and then loaded with 100 ml aliquots of the filtered supernatant. After sequential washings with 100 ml of 0.1% TFA and 100 ml of 30% methanol in 0.1% TFA, the bound material was eluted with 25 ml of 80% methanol in 0.1% TFA into a flask containing 10 μl of 300 mM PMSF. The flow rate was set at 5 ml/minute in the load and elution steps and 10 ml/minute in other steps. The eluate was concentrated to about 20 ml with a speed vac (Savant Sped-Vac SC210A). The concentrated solution was further purified with reverse phase high performance liquid chromatography (HPLC). The HPLC system consisted of Waters Delta Pak C18-100A 19 mm×30 cm column mounted onto Beckman pumps (Beckman 112 Solvent Delivery Module), which are controlled by a BioRad Chromotograph software on Windows 95. The elution gradient was created by mixture of two eluants, eluant A consisted of HPLC grade acetonitrile (ACN) and eluant B consisted of 0.1% TFA. The gradient profile was set from 25% to 45% ACN over 30 minutes. The flow rate was set at 10 ml/minute. The well-resolved peak that eluted after 40 minutes (39% ACN) was collected, frozen in liquid nitrogen and lyophilized overnight. The freeze-dried protein was stored at −20° C.

### Crystallization

Crystallization was performed by the vapor diffusion method⁵⁴ in hanging drop mode for crystal screening and in sitting drop mode for diffraction quality crystals. For hanging drops, a small bead of grease was placed on the rim of each well in the crystallization tray. Typically 500 ml of mother liquor was placed into the reservoir. The purified protein was dissolved in solution to 10 mg/ml and 2 μl of the protein solution was mixed with 2 μl of mother liquor on the silanized cover slip. The cover slip was then sealed over the well in the inverted position, such that the mother liquor and the drop on the cover slip share the same air space.

The crystals used in the structure determination were grown with a reservoir containing 0.1 M HEPES pH 7.5, 10 mM CaCl₂ and 10% w/v PEG 4000. Crystals appeared within two weeks at room temperature and reached a maximum size of 0.2 mm×0.2 mm×0.6 mm.

The invention includes a crystal comprising osteocalcin. The crystal of optionally comprises mammalian osteocalcin, such as human or porcine osteocalcin. The osteocalcin optionally comprises an amino acid sequence shown in this application or a structurally equivalent or structurally homologous sequence having at least 60%, 70%, 80%, 90% or 95% sequence identity. The crystal osteocalcin optionally comprises bound osteocalcin and hydroxyapatite. The crystal optionally comprises the following physical characteristics: diffracting to a minimum d-spacing of about 1.7 Å; a density determined by a Matthews coefficient of about Vm=2.35 Å3/Da; a solvent content of about 48%, a space group P3121 and a unit cell having dimensions a=52 Å, b=52 Å, c=35 Å, a=b=90, c=120. The crystal optionally comprises the structural coordinates presented in Table 1. The invention also includes a method for crystallizing mammalian osteocalcin, comprising crystallizing osteocalcin on a substrate by hanging drop vapour diffusion in a solution comprising buffer and precipitating solution. The substrate optionally comprises a siliconized coverslide, a plastic coverslide and a plastic microbridge. The osteocalcin is at a concentration of about: 10 mg/mL. The crystals are grown to a size of at least 0.1 mm, and, for example, to an optional maximum size of 0.2 mm×0.2 mm×0.6 mm.

### Data Collection

Single anomalous scattering (SAS) data were collected at beamline IMCA-CAT ID-17 of the Advanced Photon Source at Argonne National Laboratory. The x-ray wavelength was set at 1.7 Å to maximize the anomalous signals. Osteocalcin crystals were flash frozen in a nitrogen cold stream after transferred to a cryo-protectant solution containing 30% PEG 4000, 10 mM CaCl₂, and 0.1 M HEPES at pH 7.4. The x-ray detector, Mar Research m165 CCD, was placed 50 mm from the crystal. Data was collected in oscillation mode from 0 to 180 degree and diffraction images were recorded for each degree of rotation at 3.0 sec per frame. The crystal was kept frozen at −160° C. during data collection using the Oxford cryosystem cooling device. The intensities of the diffraction data were integrated and indexed using DENZO and reduced using SCALEPACK of the HKL package.⁵⁵ The spacegroup was P3121, with cell dimensions a=b=54 Å and c=35 Å.

### Structure Determination and Refinement

The positions of calcium and sulfur atoms were located using Bijvoet difference Patterson functions. Automated Patterson search as well as phase determination were performed with the program SOLVE.⁵⁶

Solvent flattening was carried out with the program RESOLVE.⁵⁷

The program O⁵⁸ was used for model building and rebuilding. Model building was initiated using the 2.0 Å electron density map calculated with SAS phases that were improved by solvent flattening. Refinement was carried out with the program CNS (version 1.11).⁵⁹ Positional and simulated annealing refinement with maximum likelihood targets was carried out after rigid body refinement. Iterative cycles of model building and refinement were carried out until the x-ray residual factor, R-factor, was stationary and no more information can be obtained from SigmaA-weighted⁶⁰ 2|Fo|−|Fc| electron-density map, difference map (Fo−Fc), and omit map. The model consists of all residues for the porcine osteocalcin, except a missing N-termus region (residues 1 to 12). The R-factor is 25% and the free R is 28% for reflections in the interval 30-2.0 Angstrom. 93% of the residues fall in the most favorable regions of the Ramachandran plot as defined by Procheck. Data collection and refinement statistics are summarized in Table 2. The crystal structure giving the atomic structural coordinates is given in Table 3.

The crosslinking of osteocalcin to serum albumin was performed according to the instructions provided by Molecular Probes' Protein-Protein Crosslinking Kit (P-6305).

The lysine residues in osteocalcin were thiolated with succinimidyl 3-(2-pyridyldithio)propionate (SPDP) and a thiol-reactive maleimide group is added to the lysine residues in serum albumin with succinimidyl trans-4-(maleimidylmethyl)cyclohexane-1-carboxylate (SMCC). The derived proteins were purified from SPDP and SMCC with the columns provided in the kit. The thiol group on osteocalcin was deprotected with Tris-(2-carboxyethyl)phosphine, hydrochloride (TCEP) before allowing the crosslinking reaction to occur. The crosslinked protein was purified by size exclusion chromatography. The fusion protein with the intact hydroxyapatite binding surface was isolated using a Bio-Rad hydroxyapatite column.

### Fluorescence Labeling of Osteocalcin

Osteocalcin was labeled with a highly fluorescent compound 7-aza-1-cyano-5,6-benzisoindoles to use in the disclosed drug screening assay. The procedure was performed according to the instructions provided in the ATTO-TAG kit from Molecular Probes.

### Osteocalcin-Hydroxyapatite Competitive Binding Assay

Fluoresence-labeled osteocalcin was allowed to bind and equilibrate with a hydroxyapatite suspension. Crosslinked osteocalcin or other bone drug candidates were then added. The change in fluoresence reading in the supernatant was determined with a fluorometer.

TABLE 1
Amino acid sequence of osteocalcin.

| | |
|---|---|
| | γ γ γ |
| Monkey | YLYQW LGAPA PYPDP LEPKR EVCEL NPDCD ELADH IGFQE AYRRF YGPV |
| | (SEQ ID NO: 1) |
| Rabbit | QLING QGAPA PYPDP LEPKR EVCEL NPDCD ELADQ VGLQD AYQRF YGPV |
| | (SEQ ID NO: 2) |
| Human | YLYQW LGAPA VYPDP LEPRR EVCEL NPDCD ELADH IGFQE AYRRF YGPV |
| | (SEQ ID NO: 3) |
| Cow | YLDHW LGAPA PYPDP LEPKR EVCEL NPDCD ELADH IGFQE AYRRF YGPV |
| | (SEQ ID NO: 4) |
| Pig | YLDHG LGAPA PYPDP LEPRR EVCEL NPDCD ELADH IGFQE AYRRF YGIA |
| | (SEQ ID NO: 5) |
| Sheep | YLDPG LGAPA PYPDP LEPRR EVCEL NPDCD ELADH IGFQE AYRRF YGPV |
| | (SEQ ID NO: 6) |
| Goat | YLDPG LGAPA PYPDP LEPKR EVCEL NPDCD ELADH IGFQE AYRRF YGPV |
| | (SEQ ID NO: 7) |
| Dog | YLDSG LGAPV PYPDP LEPKR EVCEL NPNCD ELADH IGFQE AYQRF YGPV |
| | (SEQ ID NO: 8) |
| Cat | YLAPG LGFPA PYPDP LEPKR EICEL NPDCD ELADH IGFQD AYRRF YGTV |
| | (SEQ ID NO: 9) |
| Wallaby | YLYQT LGAPF PYPDP QENKR EVCEL NPDCD ELADH IGFSE AYRRF YGTA |
| | (SEQ ID NO: 10) |
| Rat | YLNNG LGAPA PYPDP LEPHR EVCEL NPNCD ELADH IGFQD AYKRI YGTTV |
| | (SEQ ID NO: 11) |
| Mouse | YL GASV PSPDP LEPTR EQCEL NPACD ELSDQ YGLKT AYKRI YGITI |
| | (SEQ ID NO: 12) |
| Xenopus | SYGNN VGQGA AVGSP LESQR EVCEL NPDCD ELADH IGFQE AYRRF YGPV |
| | (SEQ ID NO: 13) |
| Emu | SFAV GSSYG AAPDP LEAQR EVCEL NPDCD ELADH IGFQE AYRRF YGPV |
| | (SEQ ID NO: 14) |
| Chicken | HYAQDS GVAGA PYPDP LEPKR EVCEL NPDCD ELADH IGFQE AYRRF YGPV |
| | (SEQ ID NO: 15) |
| Carp | AG TAPAD LTVAQ LESLK EVCEA NLACE HMMDV SGIIA AYTAY GPIPY |
| | (SEQ ID NO: 16) |
| Tetraodon | AAGE PTLQQ LESLR EVCEL NIACD EMADP AGIVA AYAAY YGPPT F |
| | (SEQ ID NO: 17) |
| Fugu | APGE PTPQQ LESLR EVCEL NIACD EMADT AGIVA AYAAY YGPPP F |
| | (SEQ ID NO: 18) |

| | |
|---|---|
| Bluegill | AAGE LTLTQ LESLR EVCEA NLACE DMMDA QGIIA AYTAY YGPIP Y |
| | (SEQ ID NO: 19) |
| Seabream | AAGQ LSLTQ LESLR EVCEL NLACE HMMDT EGIIA AYTAY YGPIP Y |
| | (SEQ ID NO: 20) |
| Swordfish | A TRAGD LTPLQ LESLR EVCEL NVSCD EMADT AGIVA AYIAY YGPIQ F |
| | [SEQ ID NO: 21) |

‘γ’ indicates a Gla residue.

TABLE 2
Statistics of data reduction and refinement.
Data collection
Space group P3₁21

| Data set | Crystal 1 | | Crystal 2 | | Combined | |
|---|---|---|---|---|---|---|
| Wavelength (Å) | 1.70 | | 1.70 | | | |
| Resolution (Å) | 27.7-2.0 | | 27.7-2.0 | | | |
| | (2.07-2.00)¹ | | (2.07-2.00)¹ | | | |
| Completeness (%) | 97.0 | (94.3)¹ | 93.1 | (92.2)¹ | 95.9 | (93.8)¹ |
| Redundancy | 17 | (≧4)¹ | 12 | (≧2)¹ | 23 | (≧5)¹ |
| R_{merge}² | 4.9 | (21.4)¹ | 4.8 | (13.3)¹ | 6.7 | (21.9)¹ |
| I/σ(I) | 14.7 | (5.35)¹ | 25.8 | (9.90)¹ | 25.1 | (16.5)¹ |
| Refinement | | | | | | |
| Unique reflection | 6230 | | | | | |
| Protein atoms | 314 | | | | | |
| Solvent atoms | 60 | | | | | |
| R-work (R_{free}) (%)³ | 25.5 | (28.3) | | | | |

| | |
|---|---|
| Average B-factor (Å²) | 37.1 |
| Deviation from ideal | |
| geometry | |
| Bonds (Å) | 0.006 |
| Angles (°) | 1.3 |
| Ramachandran plot | |
| Most favored regions | 93.3 |
| (%) | |
| Additionally allowed | 2 |
| regions (%) | |
| Generously allowed | 0 |
| regions (%) | |
| Disallowed regions | 0 |
| (%) | |

¹Highest resolution shell.
²R_{merge} = ΣₕΣᵢ |Iₕᵢ − <Iₕ>|/Σ<Iₕ>, where Iₕᵢ is the intensity of the i^{th} observation of reflection h, and <Iₕ> is the average intensity of redundant measurements of the h reflections.
³R-work = Σ∥Fₒ| − |F_{c}∥/Σ|Fₒ|, where Fₒ and F_{c} are the observed and calculated structure-factor amplitudes. R_{free} is monitored with 948 reflections excluded from refinement.

TABLE 3
Coordinate of porcine osteocalcin

| | | | |
|---|---|---|---|
| REMARK | 3 | | |
| REMARK | 3 | REFINEMENT. | |
| REMARK | 3 | PROGRAM | : CNS 1.1 |
| REMARK | 3 | AUTHORS | : BRUNGER, ADAMS, CLORE, DELANO, |
| REMARK | 3 | | GROS, GROSSE-KUNSTLEVE, JIANG, |
| REMARK | 3 | | KUSZEWSKI, NILGES, PANNU, READ, |
| REMARK | 3 | | RICE, SIMONSON, WARREN |
| REMARK | 3 | | |

| REMARK | 3 | DATA USED IN REFINEMENT. | | |
|---|---|---|---|---|
| REMARK | 3 | RESOLUTION RANGE HIGH | (ANGSTROMS) : | 2.00 |
| REMARK | 3 | RESOLUTION RANGE LOW | (ANGSTROMS) : | 27.72 |
| REMARK | 3 | DATA CUTOFF | (SIGMA(F)) : | 3.0 |
| REMARK | 3 | DATA CUTOFF HIGH | (ABS(F)) : | 462281.14 |
| REMARK | 3 | DATA CUTOFF LOW | (ABS(F)) : | 0.000000 |

| | | | | |
|---|---|---|---|---|
| REMARK | 3 | COMPLETENESS (WORKING + TEST) | (%) : | 87.7 |
| REMARK | 3 | NUMBER OF REFLECTIONS | : | 6230 |
| REMARK | 3 | | | |

| REMARK | 3 | FIT TO DATA USED IN REFINEMENT. | | |
|---|---|---|---|---|
| REMARK | 3 | CROSS-VALIDATION METHOD | : | THROUGHOUT |
| REMARK | 3 | FREE R VALUE TEST SET SELECTION | : | RANDOM |

| | | | | |
|---|---|---|---|---|
| REMARK | 3 | R VALUE | (WORKING SET) : | 0.255 |
| REMARK | 3 | FREE R VALUE | : | 0.283 |

| | | | | |
|---|---|---|---|---|
| REMARK | 3 | FREE R VALUE TEST SET SIZE | (%) : | 15.2 |
| REMARK | 3 | FREE R VALUE TEST SET COUNT | : | 948 |

| | | | | |
|---|---|---|---|---|
| REMARK | 3 | ESTIMATED ERROR OF FREE R VALUE | : | 0.009 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | : | 6 |
| REMARK | 3 | BIN RESOLUTION RANGE HIGH | (A) : | 2.00 |
| REMARK | 3 | BIN RESOLUTION RANGE LOW | (A) : | 2.13 |
| REMARK | 3 | BIN COMPLETENESS (WORKING + TEST) | (%) : | 68.6 |

| | | | | |
|---|---|---|---|---|
| REMARK | 3 | REFLECTIONS IN BIN | (WORKING SET) : | 704 |
| REMARK | 3 | BIN R VALUE | (WORKING SET) : | 0.306 |
| REMARK | 3 | BIN FREE R VALUE | : | 0.400 |

| | | | | |
|---|---|---|---|---|
| REMARK | 3 | BIN FREE R VALUE TEST SET SIZE | (%) : | 12.9 |
| REMARK | 3 | BIN FREE R VALUE TEST SET COUNT | : | 104 |

| | | | | |
|---|---|---|---|---|
| REMARK | 3 | ESTIMATED ERROR OF BIN FREE R VALUE | : | 0.039 |
| REMARK | 3 | | | |

| REMARK | 3 | NUMBER OF NON-HYDROGEN ATOMS USED IN REFINEMENT. | |
|---|---|---|---|
| REMARK | 3 | PROTEIN ATOMS | : 0 |
| REMARK | 3 | NUCLEIC ACID ATOMS | : 0 |
| REMARK | 3 | HETEROGEN ATOMS | : 0 |
| REMARK | 3 | SOLVENT ATOMS | : 0 |
| REMARK | 3 | | |
| REMARK | 3 | B VALUES. | |

| | | | | |
|---|---|---|---|---|
| REMARK | 3 | FROM WILSON PLOT | (A**2) : | 17.6 |
| REMARK | 3 | MEAN B VALUE | (OVERALL, A**2) : | 37.1 |

| REMARK | 3 | OVERALL ANISOTROPIC B VALUE. | |
|---|---|---|---|
| REMARK | 3 | B11 (A**2) : 2.05 | |
| REMARK | 3 | B22 (A**2) : 2.05 | |
| REMARK | 3 | B33 (A**2) : −4.10 | |
| REMARK | 3 | B12 (A**2) : 3.91 | |
| REMARK | 3 | B13 (A**2) : 0.00 | |
| REMARK | 3 | B23 (A**2) : 0.00 | |
| REMARK | 3 | | |
| REMARK | 3 | BULK SOLVENT MODELING. | |
| REMARK | 3 | METHOD USED | : FLAT MODEL |
| REMARK | 3 | KSOL | : 0.349568 |
| REMARK | 3 | BSOL | : 32.3123 (A**2) |
| REMARK | 3 | | |

| REMARK | 3 | ESTIMATED COORDINATE ERROR. | | |
|---|---|---|---|---|
| REMARK | 3 | ESD FROM LUZZATI PLOT | (A) : | 0.31 |
| REMARK | 3 | ESD FROM SIGMAA | (A) : | 0.27 |
| REMARK | 3 | LOW RESOLUTION CUTOFF | (A) : | 5.00 |
| REMARK | 3 | | | |
| REMARK | 3 | CROSS-VALIDATED ESTIMATED COORDINATE ERROR. | | |
| REMARK | 3 | ESD FROM C-V LUZZATI PLOT | (A) : | 0.36 |
| REMARK | 3 | ESD FROM C-V SIGMAA | (A) : | 0.35 |
| REMARK | 3 | | | |

| REMARK | 3 | RMS DEVIATIONS FROM IDEAL VALUES. | | |
|---|---|---|---|---|
| REMARK | 3 | BOND LENGTHS | (A) : | 0.006 |
| REMARK | 3 | BOND ANGLES | (DEGREES) : | 1.3 |
| REMARK | 3 | DIHEDRAL ANGLES | (DEGREES) : | 20.5 |
| REMARK | 3 | IMPROPER ANGLES | (DEGREES) : | 0.99 |
| REMARK | 3 | | | |

| REMARK | 3 | ISOTROPIC THERMAL MODEL : RESTRAINED | | | |
|---|---|---|---|---|---|
| REMARK | 3 | | | | |
| REMARK | 3 | ISOTROPIC THERMAL FACTOR RESTRAINTS. | | RMS | SIGMA |
| REMARK | 3 | MAIN-CHAIN BOND | (A**2) : | NULL | ; NULL |
| REMARK | 3 | MAIN-CHAIN ANGLE | (A**2) : | NULL | ; NULL |
| REMARK | 3 | SIDE-CHAIN BOND | (A**2) : | NULL | ; NULL |
| REMARK | 3 | SIDE-CHAIN ANGLE | (A**2) : | NULL | ; NULL |
| REMARK | 3 | | | | |
| REMARK | 3 | NCS MODEL : NONE | | | |
| REMARK | 3 | | | | |
| REMARK | 3 | NCS RESTRAINTS. | | RMS | SIGMA/WEIGHT |
| REMARK | 3 | GROUP 1 POSITIONAL | (A) : | NULL | ; NULL |
| REMARK | 3 | GROUP 1 B-FACTOR | (A**2) : | NULL | ; NULL |
| REMARK | 3 | | | | |

| REMARK | 3 | PARAMETER FILE 1 | | | | : CNS_TOPPAR/protein_rep.param | | | | | | | | | | |
|---|---|---|---|---|---|---|---|---|---|---|---|---|---|---|---|---|
| REMARK | 3 | PARAMETER FILE 2 | | | | : CNS_TOPPAR/dna-rna_rep.param | | | | | | | | | | |
| REMARK | 3 | PARAMETER FILE 3 | | | | : CNS_TOPPAR/water_rep.param | | | | | | | | | | |
| REMARK | 3 | PARAMETER FILE 4 | | | | : CNS_TOPPAR/ion.param | | | | | | | | | | |
| REMARK | 3 | TOPOLOGY FILE 1 | | | | : CNS_TOPPAR/protein.top | | | | | | | | | | |
| REMARK | 3 | TOPOLOGY FILE 2 | | | | : CNS_TOPPAR/dna-rna.top | | | | | | | | | | |
| REMARK | 3 | TOPOLOGY FILE 3 | | | | : CNS_TOPPAR/water.top | | | | | | | | | | |
| REMARK | 3 | TOPOLOGY FILE 4 | | | | : CNS_TOPPAR/ion.top | | | | | | | | | | |
| REMARK | 3 | | | | | | | | | | | | | | | |
| REMARK | 3 | OTHER REFINEMENT REMARKS: NULL | | | | | | | | | | | | | | |
| SEQRES | 1 | A | 101 | PRO | ASP | PRO | LEU | GLA | PRO | ARG | ARG | GLA | VAL | CYS | GLA | LEU |
| SEQRES | 2 | A | 101 | ASN | PRO | ASP | CYS | ASP | GLU | LEU | ALA | ASP | HIS | ILE | GLY | PHE |
| SEQRES | 3 | A | 101 | GLN | GLU | ALA | TYR | ARG | ARG | PHE | TYR | GLY | ILE | (the foregoing | | |
| corresponds to amino acids 13 to 48 SEQ ID NO: 5) | | | | | | | | | | | | | | ALA | CA2 | CA2 |
| SEQRES | 4 | A | 101 | CA2 | TIP | TIP | TIP | TIP | TIP | TIP | TIP | TIP | TIP | TIP | TIP | TIP |
| SEQRES | 5 | A | 101 | TIP | TIP | TIP | TIP | TIP | TIP | TIP | TIP | TIP | TIP | TIP | TIP | TIP |
| SEQRES | 6 | A | 101 | TIP | TIP | TIP | TIP | TIP | TIP | TIP | TIP | TIP | TIP | TIP | TIP | TIP |
| SEQRES | 7 | A | 101 | TIP | TIP | TIP | TIP | TIP | TIP | TIP | TIP | TIP | TIP | TIP | TIP | TIP |
| SEQRES | 8 | A | 101 | TIP | TIP | TIP | TIP | TIP | TIP | TIP | TIP | TIP | TIP | | | |

| | | |
|---|---|---|
| SSBOND | 1 | CYS A 23 CYS A 29 |

| | |
|---|---|
| CRYST1 | 51.491 51.491 35.389 90.00 90.00 120.00 P 31 2 1 6 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.019421 | 0.011213 | 0.000000 | 0.00000 |
| SCALE2 | 0.000000 | 0.022425 | 0.000000 | 0.00000 |
| SCALE3 | 0.000000 | 0.000000 | 0.028257 | 0.00000 |

| | | | | | | | | | | | |
|---|---|---|---|---|---|---|---|---|---|---|---|
| ATOM | 1 | CB | PRO | A | 13 | 8.383 | 28.488 | 44.434 | 1.00 | 37.68 | |
| ATOM | 2 | CG | PRO | A | 13 | 7.919 | 29.624 | 45.336 | 1.00 | 36.60 | |
| ATOM | 3 | C | PRO | A | 13 | 9.566 | 29.662 | 42.541 | 1.00 | 37.52 | |
| ATOM | 4 | O | PRO | A | 13 | 9.275 | 30.855 | 42.444 | 1.00 | 38.00 | |
| ATOM | 5 | N | PRO | A | 13 | 10.210 | 29.966 | 44.935 | 1.00 | 38.06 | |
| ATOM | 6 | CD | PRO | A | 13 | 9.196 | 30.126 | 45.995 | 1.00 | 36.47 | |
| ATOM | 7 | CA | PRO | A | 13 | 9.718 | 29.013 | 43.919 | 1.00 | 37.33 | |
| ATOM | 8 | N | ASP | A | 14 | 9.777 | 28.879 | 41.483 | 1.00 | 36.83 | |
| ATOM | 9 | CA | ASP | A | 14 | 9.671 | 29.384 | 40.116 | 1.00 | 36.13 | |
| ATOM | 10 | CB | ASP | A | 14 | 10.607 | 28.596 | 39.204 | 1.00 | 40.35 | |
| ATOM | 11 | CG | ASP | A | 14 | 10.728 | 29.211 | 37.824 | 1.00 | 43.98 | |
| ATOM | 12 | OD1 | ASP | A | 14 | 9.681 | 29.481 | 37.192 | 1.00 | 44.48 | |
| ATOM | 13 | OD2 | ASP | A | 14 | 11.874 | 29.430 | 37.371 | 1.00 | 47.64 | |
| ATOM | 14 | C | ASP | A | 14 | 8.232 | 29.268 | 39.601 | 1.00 | 33.88 | |
| ATOM | 15 | O | ASP | A | 14 | 7.721 | 28.169 | 39.413 | 1.00 | 33.66 | |
| ATOM | 16 | N | PRO | A | 15 | 7.570 | 30.409 | 39.349 | 1.00 | 30.77 | |
| ATOM | 17 | CD | PRO | A | 15 | 8.106 | 31.776 | 39.468 | 1.00 | 31.26 | |
| ATOM | 18 | CA | PRO | A | 15 | 6.189 | 30.433 | 38.856 | 1.00 | 29.44 | |
| ATOM | 19 | CB | PRO | A | 15 | 5.865 | 31.928 | 38.819 | 1.00 | 29.57 | |
| ATOM | 20 | CG | PRO | A | 15 | 7.181 | 32.562 | 38.571 | 1.00 | 28.96 | |
| ATOM | 21 | C | PRO | A | 15 | 5.990 | 29.756 | 37.497 | 1.00 | 27.79 | |
| ATOM | 22 | O | PRO | A | 15 | 4.870 | 29.396 | 37.132 | 1.00 | 23.99 | |
| ATOM | 23 | N | LEU | A | 16 | 7.088 | 29.570 | 36.763 | 1.00 | 27.93 | |
| ATOM | 24 | CA | LEU | A | 16 | 7.053 | 28.951 | 35.445 | 1.00 | 26.79 | |
| ATOM | 25 | CB | LEU | A | 16 | 8.196 | 29.502 | 34.586 | 1.00 | 28.95 | |
| ATOM | 26 | CG | LEU | A | 16 | 8.067 | 30.961 | 34.137 | 1.00 | 28.97 | |
| ATOM | 27 | CD1 | LEU | A | 16 | 9.374 | 31.410 | 33.519 | 1.00 | 31.78 | |
| ATOM | 28 | CD2 | LEU | A | 16 | 6.934 | 31.111 | 33.144 | 1.00 | 28.68 | |
| ATOM | 29 | C | LEU | A | 16 | 7.118 | 27.424 | 35.445 | 1.00 | 25.59 | |
| ATOM | 30 | O | LEU | A | 16 | 6.950 | 26.809 | 34.398 | 1.00 | 23.04 | |
| ATOM | 31 | N | CGU | A | 17 | 7.359 | 26.818 | 36.606 | 1.00 | 24.70 | |
| ATOM | 32 | CA | CGU | A | 17 | 7.453 | 25.360 | 36.702 | 1.00 | 25.21 | |
| ATOM | 33 | CB | CGU | A | 17 | 7.547 | 24.924 | 38.163 | 1.00 | 28.34 | |
| ATOM | 34 | C | CGU | A | 17 | 6.252 | 24.666 | 36.060 | 1.00 | 24.08 | |
| ATOM | 35 | O | CGU | A | 17 | 6.408 | 23.698 | 35.327 | 1.00 | 22.85 | |
| ATOM | 36 | CG | CGU | A | 17 | 8.807 | 24.090 | 38.525 | 1.00 | 29.46 | |
| ATOM | 37 | CD1 | CGU | A | 17 | 9.396 | 23.286 | 37.336 | 1.00 | 28.04 | |
| ATOM | 38 | CD2 | CGU | A | 17 | 8.411 | 23.255 | 39.740 | 1.00 | 32.29 | |
| ATOM | 39 | OE1 | CGU | A | 17 | 10.339 | 23.775 | 36.690 | 1.00 | 31.46 | |
| ATOM | 40 | OE2 | CGU | A | 17 | 8.917 | 22.160 | 37.075 | 1.00 | 26.97 | |
| ATOM | 41 | OE3 | CGU | A | 17 | 7.958 | 23.926 | 40.668 | 1.00 | 35.00 | |
| ATOM | 42 | OE4 | CGU | A | 17 | 8.527 | 22.036 | 39.780 | 1.00 | 33.69 | |
| ATOM | 43 | N | PRO | A | 18 | 5.029 | 25.135 | 36.349 | 1.00 | 23.16 | |
| ATOM | 44 | CD | PRO | A | 18 | 4.584 | 26.064 | 37.404 | 1.00 | 23.03 | |
| ATOM | 45 | CA | PRO | A | 18 | 3.884 | 24.470 | 35.727 | 1.00 | 23.07 | |
| ATOM | 46 | CB | PRO | A | 18 | 2.705 | 25.295 | 36.233 | 1.00 | 23.16 | |
| ATOM | 47 | CG | PRO | A | 18 | 3.143 | 25.654 | 37.606 | 1.00 | 21.79 | |
| ATOM | 48 | C | PRO | A | 18 | 3.985 | 24.429 | 34.196 | 1.00 | 22.93 | |
| ATOM | 49 | O | PRO | A | 18 | 3.746 | 23.388 | 33.590 | 1.00 | 23.19 | |
| ATOM | 50 | N | ARG | A | 19 | 4.339 | 25.551 | 33.573 | 1.00 | 18.14 | |
| ATOM | 51 | CA | ARG | A | 19 | 4.467 | 25.575 | 32.123 | 1.00 | 19.74 | |
| ATOM | 52 | CB | ARG | A | 19 | 4.611 | 27.011 | 31.614 | 1.00 | 19.63 | |
| ATOM | 53 | CG | ARG | A | 19 | 3.310 | 27.814 | 31.696 | 1.00 | 23.45 | |
| ATOM | 54 | CD | ARG | A | 19 | 3.424 | 29.118 | 30.948 | 1.00 | 25.21 | |
| ATOM | 55 | NE | ARG | A | 19 | 2.132 | 29.784 | 30.822 | 1.00 | 29.06 | |
| ATOM | 56 | CZ | ARG | A | 19 | 1.921 | 30.857 | 30.065 | 1.00 | 28.89 | |
| ATOM | 57 | NH1 | ARG | A | 19 | 2.919 | 31.385 | 29.368 | 1.00 | 28.53 | |
| ATOM | 58 | NH2 | ARG | A | 19 | 0.712 | 31.392 | 29.999 | 1.00 | 29.82 | |
| ATOM | 59 | C | ARG | A | 19 | 5.647 | 24.729 | 31.638 | 1.00 | 19.99 | |
| ATOM | 60 | O | ARG | A | 19 | 5.571 | 24.091 | 30.583 | 1.00 | 19.51 | |
| ATOM | 61 | N | ARG | A | 20 | 6.737 | 24.731 | 32.404 | 1.00 | 22.01 | |
| ATOM | 62 | CA | ARG | A | 20 | 7.902 | 23.943 | 32.052 | 1.00 | 22.37 | |
| ATOM | 63 | CB | ARG | A | 20 | 9.024 | 24.136 | 33.067 | 1.00 | 26.75 | |
| ATOM | 64 | CG | ARG | A | 20 | 9.586 | 25.541 | 33.115 | 1.00 | 32.44 | |
| ATOM | 65 | CD | ARG | A | 20 | 10.812 | 25.597 | 34.000 | 1.00 | 36.42 | |
| ATOM | 66 | NE | ARG | A | 20 | 11.528 | 26.853 | 33.811 | 1.00 | 43.51 | |
| ATOM | 67 | CZ | ARG | A | 20 | 12.749 | 27.098 | 34.279 | 1.00 | 47.68 | |
| ATOM | 68 | NH1 | ARG | A | 20 | 13.402 | 26.169 | 34.971 | 1.00 | 49.20 | |
| ATOM | 69 | NH2 | ARG | A | 20 | 13.323 | 28.271 | 34.045 | 1.00 | 47.89 | |
| ATOM | 70 | C | ARG | A | 20 | 7.515 | 22.468 | 32.019 | 1.00 | 24.90 | |
| ATOM | 71 | O | ARG | A | 20 | 7.956 | 21.738 | 31.130 | 1.00 | 24.00 | |
| ATOM | 72 | N | CGU | A | 21 | 6.701 | 22.022 | 32.980 | 1.00 | 24.22 | |
| ATOM | 73 | CA | CGU | A | 21 | 6.293 | 20.612 | 33.012 | 1.00 | 23.24 | |
| ATOM | 74 | CB | CGU | A | 21 | 5.506 | 20.267 | 34.289 | 1.00 | 24.58 | |
| ATOM | 75 | C | CGU | A | 21 | 5.432 | 20.293 | 31.805 | 1.00 | 23.70 | |
| ATOM | 76 | O | CGU | A | 21 | 5.561 | 19.221 | 31.216 | 1.00 | 20.30 | |
| ATOM | 77 | CG | CGU | A | 21 | 6.392 | 20.445 | 35.528 | 1.00 | 26.52 | |
| ATOM | 78 | CD1 | CGU | A | 21 | 7.353 | 19.249 | 35.754 | 1.00 | 27.96 | |
| ATOM | 79 | CD2 | CGU | A | 21 | 5.507 | 20.718 | 36.738 | 1.00 | 29.78 | |
| ATOM | 80 | OE1 | CGU | A | 21 | 8.366 | 19.406 | 36.482 | 1.00 | 27.23 | |
| ATOM | 81 | OE2 | CGU | A | 21 | 7.056 | 18.159 | 35.217 | 1.00 | 25.25 | |
| ATOM | 82 | OE3 | CGU | A | 21 | 4.695 | 21.625 | 36.586 | 1.00 | 36.91 | |
| ATOM | 83 | OE4 | CGU | A | 21 | 5.664 | 20.139 | 37.797 | 1.00 | 32.02 | |
| ATOM | 84 | N | VAL | A | 22 | 4.553 | 21.226 | 31.441 | 1.00 | 20.53 | |
| ATOM | 85 | CA | VAL | A | 22 | 3.678 | 21.031 | 30.292 | 1.00 | 21.98 | |
| ATOM | 86 | CB | VAL | A | 22 | 2.762 | 22.268 | 30.062 | 1.00 | 23.28 | |
| ATOM | 87 | CG1 | VAL | A | 22 | 2.078 | 22.180 | 28.707 | 1.00 | 24.34 | |
| ATOM | 88 | CG2 | VAL | A | 22 | 1.726 | 22.363 | 31.166 | 1.00 | 20.77 | |
| ATOM | 89 | C | VAL | A | 22 | 4.536 | 20.795 | 29.050 | 1.00 | 22.61 | |
| ATOM | 90 | O | VAL | A | 22 | 4.319 | 19.832 | 28.305 | 1.00 | 23.17 | |
| ATOM | 91 | N | CYS | A | 23 | 5.523 | 21.661 | 28.846 | 1.00 | 22.34 | |
| ATOM | 92 | CA | CYS | A | 23 | 6.423 | 21.566 | 27.699 | 1.00 | 24.86 | |
| ATOM | 93 | C | CYS | A | 23 | 7.212 | 20.248 | 27.692 | 1.00 | 25.63 | |
| ATOM | 94 | O | CYS | A | 23 | 7.306 | 19.599 | 26.656 | 1.00 | 22.02 | |
| ATOM | 95 | CB | CYS | A | 23 | 7.380 | 22.771 | 27.689 | 1.00 | 25.85 | |
| ATOM | 96 | SG | CYS | A | 23 | 8.527 | 22.921 | 26.262 | 1.00 | 30.27 | |
| ATOM | 97 | N | CGU | A | 24 | 7.761 | 19.852 | 28.842 | 1.00 | 26.69 | |
| ATOM | 98 | CA | CGU | A | 24 | 8.527 | 18.607 | 28.931 | 1.00 | 29.70 | |
| ATOM | 99 | CB | CGU | A | 24 | 8.981 | 18.304 | 30.367 | 1.00 | 26.05 | |
| ATOM | 100 | C | CGU | A | 24 | 7.665 | 17.456 | 28.476 | 1.00 | 31.08 | |
| ATOM | 101 | O | CGU | A | 24 | 8.143 | 16.541 | 27.812 | 1.00 | 32.94 | |
| ATOM | 102 | CG | CGU | A | 24 | 9.966 | 19.357 | 30.876 | 1.00 | 26.18 | |
| ATOM | 103 | CD1 | CGU | A | 24 | 11.275 | 19.290 | 30.093 | 1.00 | 24.75 | |
| ATOM | 104 | CD2 | CGU | A | 24 | 10.148 | 19.172 | 32.390 | 1.00 | 27.43 | |
| ATOM | 105 | OE1 | CGU | A | 24 | 12.023 | 18.293 | 30.233 | 1.00 | 29.79 | |
| ATOM | 106 | OE2 | CGU | A | 24 | 11.537 | 20.244 | 29.348 | 1.00 | 24.99 | |
| ATOM | 107 | OE3 | CGU | A | 24 | 9.100 | 19.190 | 33.043 | 1.00 | 28.87 | |
| ATOM | 108 | OE4 | CGU | A | 24 | 11.260 | 19.084 | 32.908 | 1.00 | 24.87 | |
| ATOM | 109 | N | LEU | A | 25 | 6.392 | 17.507 | 28.850 | 1.00 | 32.75 | |
| ATOM | 110 | CA | LEU | A | 25 | 5.445 | 16.458 | 28.506 | 1.00 | 36.12 | |
| ATOM | 111 | CB | LEU | A | 25 | 4.089 | 16.761 | 29.137 | 1.00 | 36.84 | |
| ATOM | 112 | CG | LEU | A | 25 | 3.183 | 15.549 | 29.352 | 1.00 | 36.31 | |
| ATOM | 113 | CD1 | LEU | A | 25 | 3.839 | 14.614 | 30.362 | 1.00 | 36.20 | |
| ATOM | 114 | CD2 | LEU | A | 25 | 1.821 | 15.999 | 29.854 | 1.00 | 34.69 | |
| ATOM | 115 | C | LEU | A | 25 | 5.292 | 16.308 | 26.993 | 1.00 | 36.64 | |
| ATOM | 116 | O | LEU | A | 25 | 5.064 | 15.207 | 26.490 | 1.00 | 38.56 | |
| ATOM | 117 | N | ASN | A | 26 | 5.411 | 17.422 | 26.278 | 1.00 | 37.58 | |
| ATOM | 118 | CA | ASN | A | 26 | 5.304 | 17.430 | 24.821 | 1.00 | 38.83 | |
| ATOM | 119 | CB | ASN | A | 26 | 4.709 | 18.759 | 24.340 | 1.00 | 40.62 | |
| ATOM | 120 | CG | ASN | A | 26 | 4.386 | 18.757 | 22.849 | 1.00 | 42.54 | |
| ATOM | 121 | OD1 | ASN | A | 26 | 5.213 | 18.381 | 22.014 | 1.00 | 41.62 | |
| ATOM | 122 | ND2 | ASN | A | 26 | 3.179 | 19.193 | 22.511 | 1.00 | 42.90 | |
| ATOM | 123 | C | ASN | A | 26 | 6.697 | 17.246 | 24.210 | 1.00 | 38.32 | |
| ATOM | 124 | O | ASN | A | 26 | 7.494 | 18.185 | 24.171 | 1.00 | 36.32 | |
| ATOM | 125 | N | PRO | A | 27 | 7.004 | 16.034 | 23.716 | 1.00 | 39.18 | |
| ATOM | 126 | CD | PRO | A | 27 | 6.127 | 14.868 | 23.528 | 1.00 | 39.15 | |
| ATOM | 127 | CA | PRO | A | 27 | 8.318 | 15.791 | 23.119 | 1.00 | 39.23 | |
| ATOM | 128 | CB | PRO | A | 27 | 8.135 | 14.452 | 22.401 | 1.00 | 39.42 | |
| ATOM | 129 | CG | PRO | A | 27 | 6.646 | 14.322 | 22.240 | 1.00 | 40.53 | |
| ATOM | 130 | C | PRO | A | 27 | 8.759 | 16.907 | 22.188 | 1.00 | 40.17 | |
| ATOM | 131 | O | PRO | A | 27 | 9.897 | 17.386 | 22.264 | 1.00 | 40.66 | |
| ATOM | 132 | N | ASP | A | 28 | 7.847 | 17.341 | 21.328 | 1.00 | 39.89 | |
| ATOM | 133 | CA | ASP | A | 28 | 8.149 | 18.401 | 20.383 | 1.00 | 38.18 | |
| ATOM | 134 | CB | ASP | A | 28 | 6.943 | 18.638 | 19.472 | 1.00 | 41.42 | |
| ATOM | 135 | CG | ASP | A | 28 | 6.535 | 17.386 | 18.721 | 1.00 | 41.42 | |
| ATOM | 136 | OD1 | ASP | A | 28 | 7.426 | 16.747 | 18.130 | 1.00 | 42.81 | |
| ATOM | 137 | OD2 | ASP | A | 28 | 5.333 | 17.042 | 18.720 | 1.00 | 43.97 | |
| ATOM | 138 | C | ASP | A | 28 | 8.552 | 19.697 | 21.074 | 1.00 | 37.41 | |
| ATOM | 139 | O | ASP | A | 28 | 9.459 | 20.399 | 20.611 | 1.00 | 35.62 | |
| ATOM | 140 | N | CYS | A | 29 | 7.875 | 20.022 | 22.174 | 1.00 | 34.99 | |
| ATOM | 141 | CA | CYS | A | 29 | 8.203 | 21.236 | 22.910 | 1.00 | 34.20 | |
| ATOM | 142 | C | CYS | A | 29 | 9.487 | 20.975 | 23.691 | 1.00 | 32.10 | |
| ATOM | 143 | O | CYS | A | 29 | 10.353 | 21.841 | 23.789 | 1.00 | 30.74 | |
| ATOM | 144 | CB | CYS | A | 29 | 7.080 | 21.630 | 23.891 | 1.00 | 32.50 | |
| ATOM | 145 | SG | CYS | A | 29 | 7.340 | 23.273 | 24.644 | 1.00 | 32.82 | |
| ATOM | 146 | N | ASP | A | 30 | 9.604 | 19.770 | 24.234 | 1.00 | 32.83 | |
| ATOM | 147 | CA | ASP | A | 30 | 10.776 | 19.402 | 25.014 | 1.00 | 34.15 | |
| ATOM | 148 | CB | ASP | A | 30 | 10.685 | 17.949 | 25.464 | 1.00 | 33.18 | |
| ATOM | 149 | CG | ASP | A | 30 | 11.714 | 17.607 | 26.523 | 1.00 | 32.22 | |
| ATOM | 150 | OD1 | ASP | A | 30 | 12.621 | 18.428 | 26.752 | 1.00 | 32.53 | |
| ATOM | 151 | OD2 | ASP | A | 30 | 11.608 | 16.524 | 27.125 | 1.00 | 31.78 | |
| ATOM | 152 | C | ASP | A | 30 | 12.026 | 19.580 | 24.177 | 1.00 | 36.29 | |
| ATOM | 153 | O | ASP | A | 30 | 12.937 | 20.322 | 24.544 | 1.00 | 34.50 | |
| ATOM | 154 | N | GLU | A | 31 | 12.056 | 18.885 | 23.045 | 1.00 | 39.34 | |
| ATOM | 155 | CA | GLU | A | 31 | 13.186 | 18.954 | 22.135 | 1.00 | 40.16 | |
| ATOM | 156 | CB | GLU | A | 31 | 12.901 | 18.124 | 20.883 | 1.00 | 42.69 | |
| ATOM | 157 | CG | GLU | A | 31 | 13.972 | 18.251 | 19.813 | 1.00 | 45.38 | |
| ATOM | 158 | CD | GLU | A | 31 | 15.358 | 17.952 | 20.345 | 1.00 | 45.54 | |
| ATOM | 159 | OE1 | GLU | A | 31 | 15.566 | 16.825 | 20.847 | 1.00 | 44.75 | |
| ATOM | 160 | OE2 | GLU | A | 31 | 16.230 | 18.845 | 20.260 | 1.00 | 44.69 | |
| ATOM | 161 | C | GLU | A | 31 | 13.483 | 20.394 | 21.744 | 1.00 | 40.61 | |
| ATOM | 162 | O | GLU | A | 31 | 14.609 | 20.863 | 21.886 | 1.00 | 41.73 | |
| ATOM | 163 | N | LEU | A | 32 | 12.464 | 21.100 | 21.269 | 1.00 | 40.32 | |
| ATOM | 164 | CA | LEU | A | 32 | 12.638 | 22.483 | 20.846 | 1.00 | 40.10 | |
| ATOM | 165 | CB | LEU | A | 32 | 11.301 | 23.068 | 20.367 | 1.00 | 39.37 | |
| ATOM | 166 | CG | LEU | A | 32 | 11.349 | 24.312 | 19.462 | 1.00 | 40.36 | |
| ATOM | 167 | CD1 | LEU | A | 32 | 9.943 | 24.629 | 18.995 | 1.00 | 39.47 | |
| ATOM | 168 | CD2 | LEU | A | 32 | 11.946 | 25.520 | 20.187 | 1.00 | 38.65 | |
| ATOM | 169 | C | LEU | A | 32 | 13.205 | 23.351 | 21.958 | 1.00 | 40.12 | |
| ATOM | 170 | O | LEU | A | 32 | 14.023 | 24.237 | 21.702 | 1.00 | 42.39 | |
| ATOM | 171 | N | ALA | A | 33 | 12.767 | 23.102 | 23.190 | 1.00 | 39.67 | |
| ATOM | 172 | CA | ALA | A | 33 | 13.225 | 23.877 | 24.346 | 1.00 | 39.26 | |
| ATOM | 173 | CB | ALA | A | 33 | 12.600 | 23.326 | 25.630 | 1.00 | 37.33 | |
| ATOM | 174 | C | ALA | A | 33 | 14.746 | 23.914 | 24.481 | 1.00 | 38.24 | |
| ATOM | 175 | O | ALA | A | 33 | 15.317 | 24.939 | 24.866 | 1.00 | 39.05 | |
| ATOM | 176 | N | ASP | A | 34 | 15.400 | 22.799 | 24.170 | 1.00 | 39.56 | |
| ATOM | 177 | CA | ASP | A | 34 | 16.857 | 22.723 | 24.258 | 1.00 | 40.96 | |
| ATOM | 178 | CB | ASP | A | 34 | 17.352 | 21.300 | 23.976 | 1.00 | 40.20 | |
| ATOM | 179 | CG | ASP | A | 34 | 17.006 | 20.327 | 25.083 | 1.00 | 38.93 | |
| ATOM | 180 | OD1 | ASP | A | 34 | 16.981 | 20.742 | 26.262 | 1.00 | 41.79 | |
| ATOM | 181 | OD2 | ASP | A | 34 | 16.777 | 19.140 | 24.778 | 1.00 | 37.45 | |
| ATOM | 182 | C | ASP | A | 34 | 17.570 | 23.672 | 23.301 | 1.00 | 42.49 | |
| ATOM | 183 | O | ASP | A | 34 | 18.752 | 23.962 | 23.482 | 1.00 | 44.27 | |
| ATOM | 184 | N | HIS | A | 35 | 16.859 | 24.168 | 22.295 | 1.00 | 42.65 | |
| ATOM | 185 | CA | HIS | A | 35 | 17.477 | 25.040 | 21.310 | 1.00 | 43.28 | |
| ATOM | 186 | CB | HIS | A | 35 | 17.078 | 24.570 | 19.911 | 1.00 | 43.83 | |
| ATOM | 187 | CG | HIS | A | 35 | 17.309 | 23.108 | 19.691 | 1.00 | 43.04 | |
| ATOM | 188 | CD2 | HIS | A | 35 | 16.455 | 22.056 | 19.723 | 1.00 | 44.56 | |
| ATOM | 189 | ND1 | HIS | A | 35 | 18.563 | 22.572 | 19.492 | 1.00 | 44.90 | |
| ATOM | 190 | CE1 | HIS | A | 35 | 18.472 | 21.256 | 19.415 | 1.00 | 44.90 | |
| ATOM | 191 | NE2 | HIS | A | 35 | 17.201 | 20.918 | 19.554 | 1.00 | 42.77 | |
| ATOM | 192 | C | HIS | A | 35 | 17.175 | 26.519 | 21.478 | 1.00 | 44.83 | |
| ATOM | 193 | O | HIS | A | 35 | 18.097 | 27.330 | 21.567 | 1.00 | 44.27 | |
| ATOM | 194 | N | ILE | A | 36 | 15.895 | 26.878 | 21.523 | 1.00 | 45.92 | |
| ATOM | 195 | CA | ILE | A | 36 | 15.529 | 28.283 | 21.676 | 1.00 | 47.35 | |
| ATOM | 196 | CB | ILE | A | 36 | 14.513 | 28.709 | 20.571 | 1.00 | 49.18 | |
| ATOM | 197 | CG2 | ILE | A | 36 | 13.106 | 28.847 | 21.143 | 1.00 | 49.20 | |
| ATOM | 198 | CG1 | ILE | A | 36 | 14.986 | 30.014 | 19.921 | 1.00 | 49.48 | |
| ATOM | 199 | CD1 | ILE | A | 36 | 15.256 | 31.136 | 20.902 | 1.00 | 51.35 | |
| ATOM | 200 | C | ILE | A | 36 | 14.989 | 28.622 | 23.073 | 1.00 | 46.83 | |
| ATOM | 201 | O | ILE | A | 36 | 14.593 | 29.756 | 23.345 | 1.00 | 46.83 | |
| ATOM | 202 | N | GLY | A | 37 | 14.993 | 27.639 | 23.966 | 1.00 | 46.04 | |
| ATOM | 203 | CA | GLY | A | 37 | 14.511 | 27.885 | 25.316 | 1.00 | 45.48 | |
| ATOM | 204 | C | GLY | A | 37 | 13.082 | 27.430 | 25.553 | 1.00 | 43.62 | |
| ATOM | 205 | O | GLY | A | 37 | 12.346 | 27.151 | 24.610 | 1.00 | 43.67 | |
| ATOM | 206 | N | PHE | A | 38 | 12.694 | 27.360 | 26.821 | 1.00 | 42.00 | |
| ATOM | 207 | CA | PHE | A | 38 | 11.354 | 26.932 | 27.200 | 1.00 | 42.44 | |
| ATOM | 208 | CB | PHE | A | 38 | 11.356 | 26.492 | 28.676 | 1.00 | 42.09 | |
| ATOM | 209 | CG | PHE | A | 38 | 10.116 | 26.875 | 29.427 | 1.00 | 43.59 | |
| ATOM | 210 | CD1 | PHE | A | 38 | 8.890 | 26.284 | 29.136 | 1.00 | 42.94 | |
| ATOM | 211 | CD2 | PHE | A | 38 | 10.167 | 27.869 | 30.400 | 1.00 | 44.79 | |
| ATOM | 212 | CE1 | PHE | A | 38 | 7.730 | 26.685 | 29.797 | 1.00 | 42.28 | |
| ATOM | 213 | CE2 | PHE | A | 38 | 9.016 | 28.275 | 31.063 | 1.00 | 44.67 | |
| ATOM | 214 | CZ | PHE | A | 38 | 7.795 | 27.680 | 30.760 | 1.00 | 43.27 | |
| ATOM | 215 | C | PHE | A | 38 | 10.266 | 27.986 | 26.950 | 1.00 | 41.79 | |
| ATOM | 216 | O | PHE | A | 38 | 9.248 | 27.685 | 26.326 | 1.00 | 40.84 | |
| ATOM | 217 | N | GLN | A | 39 | 10.475 | 29.213 | 27.418 | 1.00 | 42.13 | |
| ATOM | 218 | CA | GLN | A | 39 | 9.472 | 30.266 | 27.242 | 1.00 | 44.46 | |
| ATOM | 219 | CB | GLN | A | 39 | 9.880 | 31.543 | 27.985 | 1.00 | 46.30 | |
| ATOM | 220 | CG | GLN | A | 39 | 10.027 | 31.369 | 29.488 | 1.00 | 48.51 | |
| ATOM | 221 | CD | GLN | A | 39 | 10.066 | 32.693 | 30.229 | 1.00 | 50.88 | |
| ATOM | 222 | OE1 | GLN | A | 39 | 9.079 | 33.434 | 30.251 | 1.00 | 50.83 | |
| ATOM | 223 | NE2 | GLN | A | 39 | 11.208 | 32.998 | 30.843 | 1.00 | 51.78 | |
| ATOM | 224 | C | GLN | A | 39 | 9.177 | 30.607 | 25.786 | 1.00 | 45.01 | |
| ATOM | 225 | O | GLN | A | 39 | 8.075 | 31.048 | 25.457 | 1.00 | 45.18 | |
| ATOM | 226 | N | GLU | A | 40 | 10.155 | 30.407 | 24.912 | 1.00 | 45.57 | |
| ATOM | 227 | CA | GLU | A | 40 | 9.955 | 30.700 | 23.500 | 1.00 | 44.79 | |
| ATOM | 228 | CB | GLU | A | 40 | 11.281 | 31.096 | 22.846 | 1.00 | 46.36 | |
| ATOM | 229 | CG | GLU | A | 40 | 11.131 | 31.647 | 21.438 | 1.00 | 48.30 | |
| ATOM | 230 | CD | GLU | A | 40 | 10.174 | 32.820 | 21.374 | 1.00 | 49.05 | |
| ATOM | 231 | OE1 | GLU | A | 40 | 10.392 | 33.804 | 22.116 | 1.00 | 50.84 | |
| ATOM | 232 | OE2 | GLU | A | 40 | 9.204 | 32.755 | 20.583 | 1.00 | 48.60 | |
| ATOM | 233 | C | GLU | A | 40 | 9.368 | 29.475 | 22.808 | 1.00 | 43.43 | |
| ATOM | 234 | O | GLU | A | 40 | 8.620 | 29.592 | 21.833 | 1.00 | 42.89 | |
| ATOM | 235 | N | ALA | A | 41 | 9.702 | 28.299 | 23.327 | 1.00 | 41.16 | |
| ATOM | 236 | CA | ALA | A | 41 | 9.201 | 27.051 | 22.765 | 1.00 | 39.01 | |
| ATOM | 237 | CB | ALA | A | 41 | 10.012 | 25.877 | 23.275 | 1.00 | 37.48 | |
| ATOM | 238 | C | ALA | A | 41 | 7.740 | 26.877 | 23.146 | 1.00 | 38.28 | |
| ATOM | 239 | O | ALA | A | 41 | 6.915 | 26.485 | 22.317 | 1.00 | 38.27 | |
| ATOM | 240 | N | TYR | A | 42 | 7.422 | 27.163 | 24.406 | 1.00 | 36.31 | |
| ATOM | 241 | CA | TYR | A | 42 | 6.048 | 27.041 | 24.880 | 1.00 | 34.56 | |
| ATOM | 242 | CB | TYR | A | 42 | 5.937 | 27.497 | 26.340 | 1.00 | 32.46 | |
| ATOM | 243 | CG | TYR | A | 42 | 4.592 | 27.197 | 26.980 | 1.00 | 29.55 | |
| ATOM | 244 | CD1 | TYR | A | 42 | 4.343 | 25.966 | 27.591 | 1.00 | 27.43 | |
| ATOM | 245 | CE1 | TYR | A | 42 | 3.090 | 25.673 | 28.151 | 1.00 | 27.03 | |
| ATOM | 246 | CD2 | TYR | A | 42 | 3.561 | 28.135 | 26.950 | 1.00 | 28.09 | |
| ATOM | 247 | CE2 | TYR | A | 42 | 2.308 | 27.852 | 27.504 | 1.00 | 28.26 | |
| ATOM | 248 | CZ | TYR | A | 42 | 2.082 | 26.627 | 28.103 | 1.00 | 28.64 | |
| ATOM | 249 | OH | TYR | A | 42 | 0.843 | 26.362 | 28.646 | 1.00 | 30.72 | |
| ATOM | 250 | C | TYR | A | 42 | 5.173 | 27.923 | 23.991 | 1.00 | 36.12 | |
| ATOM | 251 | O | TYR | A | 42 | 4.152 | 27.475 | 23.471 | 1.00 | 36.61 | |
| ATOM | 252 | N | ARG | A | 43 | 5.591 | 29.174 | 23.813 | 1.00 | 36.78 | |
| ATOM | 253 | CA | ARG | A | 43 | 4.863 | 30.127 | 22.983 | 1.00 | 40.45 | |
| ATOM | 254 | CB | ARG | A | 43 | 5.614 | 31.462 | 22.918 | 1.00 | 42.48 | |
| ATOM | 255 | CG | ARG | A | 43 | 5.062 | 32.408 | 21.865 | 1.00 | 46.79 | |
| ATOM | 256 | CD | ARG | A | 43 | 6.001 | 33.559 | 21.565 | 1.00 | 49.17 | |
| ATOM | 257 | NE | ARG | A | 43 | 5.896 | 33.961 | 20.166 | 1.00 | 52.05 | |
| ATOM | 258 | CZ | ARG | A | 43 | 6.187 | 33.163 | 19.141 | 1.00 | 53.09 | |
| ATOM | 259 | NH1 | ARG | A | 43 | 6.603 | 31.924 | 19.363 | 1.00 | 54.07 | |
| ATOM | 260 | NH2 | ARG | A | 43 | 6.056 | 33.595 | 17.892 | 1.00 | 52.56 | |
| ATOM | 261 | C | ARG | A | 43 | 4.640 | 29.610 | 21.565 | 1.00 | 41.08 | |
| ATOM | 262 | O | ARG | A | 43 | 3.581 | 29.833 | 20.980 | 1.00 | 40.43 | |
| ATOM | 263 | N | ARG | A | 44 | 5.643 | 28.925 | 21.017 | 1.00 | 41.91 | |
| ATOM | 264 | CA | ARG | A | 44 | 5.566 | 28.380 | 19.668 | 1.00 | 41.07 | |
| ATOM | 265 | CB | ARG | A | 44 | 6.915 | 27.782 | 19.250 | 1.00 | 45.64 | |
| ATOM | 266 | CG | ARG | A | 44 | 7.861 | 28.777 | 18.576 | 1.00 | 48.95 | |
| ATOM | 267 | CD | ARG | A | 44 | 7.141 | 29.513 | 17.448 | 1.00 | 53.17 | |
| ATOM | 268 | NE | ARG | A | 44 | 6.442 | 28.585 | 16.559 | 1.00 | 57.69 | |
| ATOM | 269 | CZ | ARG | A | 44 | 5.469 | 28.935 | 15.720 | 1.00 | 59.72 | |
| ATOM | 270 | NH1 | ARG | A | 44 | 4.895 | 28.020 | 14.951 | 1.00 | 60.60 | |
| ATOM | 271 | NH2 | ARG | A | 44 | 5.061 | 30.197 | 15.655 | 1.00 | 60.94 | |
| ATOM | 272 | C | ARG | A | 44 | 4.478 | 27.334 | 19.488 | 1.00 | 40.12 | |
| ATOM | 273 | O | ARG | A | 44 | 3.787 | 27.334 | 18.476 | 1.00 | 38.24 | |
| ATOM | 274 | N | PHE | A | 45 | 4.324 | 26.446 | 20.467 | 1.00 | 39.96 | |
| ATOM | 275 | CA | PHE | A | 45 | 3.312 | 25.393 | 20.387 | 1.00 | 38.49 | |
| ATOM | 276 | CB | PHE | A | 45 | 3.824 | 24.101 | 21.026 | 1.00 | 40.88 | |
| ATOM | 277 | CG | PHE | A | 45 | 4.808 | 23.342 | 20.184 | 1.00 | 43.51 | |
| ATOM | 278 | CD1 | PHE | A | 45 | 6.129 | 23.759 | 20.082 | 1.00 | 44.81 | |
| ATOM | 279 | CD2 | PHE | A | 45 | 4.413 | 22.188 | 19.513 | 1.00 | 45.28 | |
| ATOM | 280 | CE1 | PHE | A | 45 | 7.051 | 23.032 | 19.327 | 1.00 | 45.40 | |
| ATOM | 281 | CE2 | PHE | A | 45 | 5.322 | 21.455 | 18.756 | 1.00 | 46.39 | |
| ATOM | 282 | CZ | PHE | A | 45 | 6.648 | 21.879 | 18.664 | 1.00 | 46.57 | |
| ATOM | 283 | C | PHE | A | 45 | 1.969 | 25.721 | 21.045 | 1.00 | 36.87 | |
| ATOM | 284 | O | PHE | A | 45 | 0.969 | 25.065 | 20.761 | 1.00 | 36.85 | |
| ATOM | 285 | N | TYR | A | 46 | 1.935 | 26.714 | 21.927 | 1.00 | 34.86 | |
| ATOM | 286 | CA | TYR | A | 46 | 0.694 | 27.025 | 22.624 | 1.00 | 33.03 | |
| ATOM | 287 | CB | TYR | A | 46 | 0.827 | 26.598 | 24.081 | 1.00 | 29.21 | |
| ATOM | 288 | CG | TYR | A | 46 | 1.202 | 25.154 | 24.222 | 1.00 | 28.43 | |
| ATOM | 289 | CD1 | TYR | A | 46 | 2.446 | 24.773 | 24.736 | 1.00 | 28.99 | |
| ATOM | 290 | CE1 | TYR | A | 46 | 2.790 | 23.427 | 24.860 | 1.00 | 25.13 | |
| ATOM | 291 | CD2 | TYR | A | 46 | 0.321 | 24.156 | 23.824 | 1.00 | 27.13 | |
| ATOM | 292 | CE2 | TYR | A | 46 | 0.656 | 22.822 | 23.936 | 1.00 | 27.50 | |
| ATOM | 293 | CZ | TYR | A | 46 | 1.888 | 22.461 | 24.457 | 1.00 | 26.34 | |
| ATOM | 294 | OH | TYR | A | 46 | 2.174 | 21.125 | 24.613 | 1.00 | 27.54 | |
| ATOM | 295 | C | TYR | A | 46 | 0.240 | 28.472 | 22.552 | 1.00 | 34.15 | |
| ATOM | 296 | O | TYR | A | 46 | −0.939 | 28.766 | 22.749 | 1.00 | 35.46 | |
| ATOM | 297 | N | GLY | A | 47 | 1.170 | 29.378 | 22.278 | 1.00 | 33.49 | |
| ATOM | 298 | CA | GLY | A | 47 | 0.802 | 30.773 | 22.189 | 1.00 | 33.72 | |
| ATOM | 299 | C | GLY | A | 47 | −0.027 | 31.059 | 20.951 | 1.00 | 36.70 | |
| ATOM | 300 | O | GLY | A | 47 | −0.146 | 30.229 | 20.044 | 1.00 | 34.81 | |
| ATOM | 301 | N | ILE | A | 48 | −0.634 | 32.237 | 20.925 | 1.00 | 37.59 | |
| ATOM | 302 | CA | ILE | A | 48 | −1.421 | 32.647 | 19.783 | 1.00 | 40.72 | |
| ATOM | 303 | CB | ILE | A | 48 | −2.751 | 33.295 | 20.202 | 1.00 | 40.52 | |
| ATOM | 304 | CG2 | ILE | A | 48 | −3.465 | 33.866 | 18.972 | 1.00 | 39.46 | |
| ATOM | 305 | CG1 | ILE | A | 48 | −3.629 | 32.260 | 20.905 | 1.00 | 40.08 | |
| ATOM | 306 | CD1 | ILE | A | 48 | −4.898 | 32.839 | 21.493 | 1.00 | 41.90 | |
| ATOM | 307 | C | ILE | A | 48 | −0.549 | 33.679 | 19.102 | 1.00 | 43.37 | |
| ATOM | 308 | O | ILE | A | 48 | −0.447 | 34.817 | 19.561 | 1.00 | 44.91 | |
| ATOM | 309 | N | ALA | A | 49 | 0.100 | 33.262 | 18.021 | 1.00 | 45.31 | |
| ATOM | 310 | CA | ALA | A | 49 | 0.999 | 34.130 | 17.266 | 1.00 | 47.31 | |
| ATOM | 311 | CB | ALA | A | 49 | 0.427 | 35.551 | 17.179 | 1.00 | 48.09 | |
| ATOM | 312 | C | ALA | A | 49 | 2.381 | 34.152 | 17.921 | 1.00 | 46.55 | |
| ATOM | 313 | O | ALA | A | 49 | 2.587 | 33.374 | 18.881 | 1.00 | 45.60 | |
| ATOM | 314 | OXT | ALA | A | 49 | 3.237 | 34.938 | 17.462 | 1.00 | 45.24 | |
| ATOM | 315 | CA + 2 | CA2 | A | 1 | 13.077 | 17.433 | 32.271 | 1.00 | 22.23 | C |
| ATOM | 316 | CA + 2 | CA2 | A | 2 | 13.835 | 18.867 | 28.887 | 1.00 | 30.50 | C |
| ATOM | 317 | CA + 2 | CA2 | A | 3 | 10.897 | 18.813 | 35.385 | 1.00 | 50.79 | C |
| ATOM | 318 | OH2 | TIP | A | 1 | 5.850 | 30.876 | 28.875 | 1.00 | 26.66 | S |
| ATOM | 319 | OH2 | TIP | A | 2 | 13.387 | 22.461 | 33.530 | 1.00 | 24.93 | S |
| ATOM | 320 | OH2 | TIP | A | 3 | 2.021 | 19.160 | 26.919 | 1.00 | 35.80 | S |
| ATOM | 321 | OH2 | TIP | A | 4 | 5.863 | 14.666 | 19.011 | 1.00 | 38.16 | S |
| ATOM | 322 | OH2 | TIP | A | 5 | 10.578 | 15.304 | 29.567 | 1.00 | 23.15 | S |
| ATOM | 323 | OH2 | TIP | A | 6 | 5.020 | 20.563 | 40.636 | 1.00 | 44.02 | S |
| ATOM | 324 | OH2 | TIP | A | 7 | 2.823 | 22.144 | 38.546 | 1.00 | 36.74 | S |
| ATOM | 325 | OH2 | TIP | A | 8 | 10.434 | 22.631 | 29.604 | 1.00 | 25.89 | S |
| ATOM | 326 | OH2 | TIP | A | 9 | 6.522 | 15.691 | 36.473 | 1.00 | 27.82 | S |
| ATOM | 327 | OH2 | TIP | A | 10 | 2.927 | 29.395 | 38.649 | 1.00 | 33.09 | S |
| ATOM | 328 | OH2 | TIP | A | 11 | 8.208 | 35.765 | 32.338 | 1.00 | 47.23 | S |
| ATOM | 329 | OH2 | TIP | A | 12 | 14.353 | 36.470 | 34.820 | 1.00 | 66.90 | S |
| ATOM | 330 | OH2 | TIP | A | 13 | 3.807 | 28.482 | 34.824 | 1.00 | 24.88 | S |
| ATOM | 331 | OH2 | TIP | A | 14 | 11.624 | 15.358 | 31.822 | 1.00 | 24.92 | S |
| ATOM | 332 | OH2 | TIP | A | 15 | 13.763 | 16.798 | 28.667 | 1.00 | 29.47 | S |
| ATOM | 333 | OH2 | TIP | A | 16 | 6.350 | 16.973 | 32.340 | 1.00 | 37.83 | S |
| ATOM | 334 | OH2 | TIP | A | 17 | 9.425 | 33.464 | 43.095 | 1.00 | 58.60 | S |
| ATOM | 335 | OH2 | TIP | A | 18 | 3.199 | 34.744 | 28.589 | 1.00 | 35.94 | S |
| ATOM | 336 | OH2 | TIP | A | 19 | 13.597 | 33.847 | 46.467 | 1.00 | 45.84 | S |
| ATOM | 337 | OH2 | TIP | A | 20 | 10.474 | 21.054 | 34.739 | 1.00 | 25.48 | S |
| ATOM | 338 | OH2 | TIP | A | 21 | 8.008 | 14.321 | 26.270 | 1.00 | 36.62 | S |
| ATOM | 339 | OH2 | TIP | A | 22 | 5.694 | 31.583 | 26.473 | 1.00 | 54.83 | S |
| ATOM | 340 | OH2 | TIP | A | 23 | 6.216 | 35.113 | 27.449 | 1.00 | 38.82 | S |
| ATOM | 341 | OH2 | TIP | A | 24 | 16.203 | 18.688 | 27.720 | 1.00 | 28.10 | S |
| ATOM | 342 | OH2 | TIP | A | 25 | 8.186 | 14.327 | 30.477 | 1.00 | 49.44 | S |
| ATOM | 343 | OH2 | TIP | A | 26 | 8.625 | 16.477 | 33.868 | 1.00 | 48.13 | S |
| ATOM | 344 | OH2 | TIP | A | 27 | 5.125 | 11.770 | 28.038 | 1.00 | 39.55 | S |
| ATOM | 345 | OH2 | TIP | A | 28 | 2.083 | 16.119 | 25.781 | 1.00 | 38.18 | S |
| ATOM | 346 | OH2 | TIP | A | 29 | 15.462 | 16.714 | 24.789 | 1.00 | 42.90 | S |
| ATOM | 347 | OH2 | TIP | A | 30 | 13.510 | 28.016 | 31.338 | 1.00 | 58.36 | S |
| ATOM | 348 | OH2 | TIP | A | 31 | 3.415 | 31.464 | 25.271 | 1.00 | 46.78 | S |
| ATOM | 349 | OH2 | TIP | A | 33 | −0.797 | 33.535 | 23.539 | 1.00 | 27.00 | S |
| ATOM | 350 | OH2 | TIP | A | 34 | −1.094 | 29.634 | 25.805 | 1.00 | 39.49 | S |
| ATOM | 351 | OH2 | TIP | A | 35 | 1.137 | 31.111 | 26.310 | 1.00 | 31.80 | S |
| ATOM | 352 | OH2 | TIP | A | 36 | 1.407 | 37.001 | 28.210 | 1.00 | 37.32 | S |
| ATOM | 353 | OH2 | TIP | A | 37 | 0.970 | 33.425 | 27.520 | 1.00 | 52.33 | S |
| ATOM | 354 | OH2 | TIP | A | 38 | −2.315 | 31.723 | 29.953 | 1.00 | 44.23 | S |
| ATOM | 355 | OH2 | TIP | A | 39 | 4.757 | 17.423 | 38.879 | 1.00 | 34.26 | S |
| ATOM | 356 | OH2 | TIP | A | 40 | 3.611 | 36.978 | 27.027 | 1.00 | 33.99 | S |
| ATOM | 357 | OH2 | TIP | A | 41 | 10.313 | 14.495 | 25.452 | 1.00 | 40.66 | S |
| ATOM | 358 | OH2 | TIP | A | 42 | 1.979 | 18.616 | 37.760 | 1.00 | 34.25 | S |
| ATOM | 359 | OH2 | TIP | A | 43 | 5.964 | 18.909 | 16.412 | 1.00 | 39.77 | S |
| ATOM | 360 | OH2 | TIP | A | 44 | 1.860 | 21.461 | 34.673 | 1.00 | 32.95 | S |
| ATOM | 361 | OH2 | TIP | A | 45 | 11.462 | 18.113 | 17.461 | 1.00 | 52.62 | S |
| ATOM | 362 | OH2 | TIP | A | 46 | 13.926 | 20.627 | 27.271 | 1.00 | 29.62 | S |
| ATOM | 363 | OH2 | TIP | A | 47 | 19.590 | 28.299 | 19.078 | 1.00 | 43.97 | S |
| ATOM | 364 | OH2 | TIP | A | 48 | 16.240 | 23.471 | 27.700 | 1.00 | 48.79 | S |
| ATOM | 365 | OH2 | TIP | A | 49 | 4.036 | 16.714 | 34.084 | 1.00 | 48.66 | S |
| ATOM | 366 | OH2 | TIP | A | 50 | 12.966 | 33.075 | 18.816 | 1.00 | 57.37 | S |
| ATOM | 367 | OH2 | TIP | A | 51 | 4.126 | 14.417 | 36.341 | 1.00 | 40.73 | S |
| ATOM | 368 | OH2 | TIP | A | 52 | 11.703 | 37.543 | 30.651 | 1.00 | 36.00 | S |
| ATOM | 369 | OH2 | TIP | A | 53 | 2.747 | 18.823 | 35.170 | 1.00 | 50.30 | S |
| ATOM | 370 | OH2 | TIP | A | 54 | 0.279 | 24.293 | 38.899 | 1.00 | 43.36 | S |
| ATOM | 371 | OH2 | TIP | A | 55 | 5.228 | 23.553 | 41.559 | 1.00 | 42.02 | S |
| ATOM | 372 | OH2 | TIP | A | 56 | 5.298 | 21.833 | 43.473 | 1.00 | 41.96 | S |
| ATOM | 373 | OH2 | TIP | A | 57 | −2.985 | 34.432 | 24.688 | 1.00 | 37.28 | S |
| ATOM | 374 | OH2 | TIP | A | 58 | 9.768 | 32.886 | 36.715 | 1.00 | 30.34 | S |
| ATOM | 375 | OH2 | TIP | A | 59 | 11.644 | 31.779 | 15.209 | 1.00 | 38.45 | S |
| ATOM | 376 | OH2 | TIP | A | 60 | 13.181 | 22.613 | 29.210 | 1.00 | 35.43 | S |
| ATOM | 377 | OH2 | TIP | A | 63 | 3.510 | 13.299 | 33.052 | 1.00 | 44.76 | S |
| ATOM | 378 | OH2 | TIP | A | 64 | 23.246 | 30.853 | 39.777 | 1.00 | 53.00 | S |
| TER | | | | | | | | | | | |
| END | | | | | | | | | | | |

### REFERENCES

1. Driessens, F. C. M. & Verbeeck, R. M. H. Biominerals. 179-219 (CRC Press, Boca Raton, 1990).
2. Weiner, S. & Wagner, H. D. The material bone: structure-mechanical function relations. Annu. Rev. Mater. Sci. 28, 271-298 (1998).
3. Weiner, S., Traub, W. & Wagner, H. D. Lamellar Bone: Structure-Function Relations. J Struct Biol 126, 241-255 (1999).
4. Ziv, V., Wagner, H. D. & Weiner, S. Microstructure-Microhardness relations in parallel-fibered and lamellar bone. Bone 18, 417-428 (1996).
5. Compton, J. E. Bone marrow and bone: a functional unit. J. Endocrinology 173, 387-394 (2002).
6. Hauschka, P. V. & Carr, S. A. Calcium-dependent α-heical structure in osteocalcin. Biochemistry 21, 2538-2547 (1982).
7. Lian, J. B., Roufosse, A. H., Reit, B. & Glimcher, M. J. Concentration of osteocalcin and phosphoprotein as a function of mineral content and age in cortical bone. Calcif. Tissue Int 34, 82-87 (1982).
8. Conn, K. M. & Termine, J. D. Matrix protein profiles in calf bone development. Bone 6, 1985 (1985).
9. Poser, J. W. & Price, P.A. A Method for Decarboxylation of γ-Carboxyglutamic Acid in Proteins. J Biol Chem 254, 431-436 (1979).
10. Gallop, P. M., Lian, J. B. & Hauschka, P. V. Carboxylated calcium-binding proteins and vitamin K. N. Engl. J. Med. 302, 1460-1466 (1980).
11. Atkinson, R. A. et al. Conformational studies of osteocalcin in solution. Eur J Biochem 232, 515-521 (1995).
12. Isbell, D. T., Du, S., Schroering, A. G., Colombo, G. & Shelling, J. G. Metal Ion Binding to Dog Osteocalcin Studied by ¹H NMR Spectroscopy. Biochemistry 32, 11352-11362 (1993).
13. Hauschka, P. V., Lian, J. B., Cole, D. E. C. & Gundberg, C. M. Osteocalcin and Matrix Gla Protein: Vitamin K-Dependent Proteins in Bone. Physiol Rev 69, 990-1047 (1989).
14. Hauschka, P. V. & Reid, M. L. Vitamin K dependence of a calcium binding protein containing gamma-carboxyglutamic acid in chicken bone. J. Biol. Chem. 253, 237-243 (1978).
15. Price, P. A. & Willamson, M. K. Effects of warfarin on bone: studies on the vitamin K-dependent protein of rat bone. J. Biol. Cheml. 256, 12754-12759 (1981).
16. Pastoureau, P., Vergnaud, P., Meunier, P. J. & Delmas, P. D. Osteopenia and bone-remodeling abnormalities in warfarin-treated lambs. J Bone Miner Res 8, 1417-1426 (1993).
17. Mizuguchi, M., Fujisawa, R., Nara, M., Nitta, K. & Kawano, K. Fourier-Transform Infrared Spectroscopic Study of Ca²⁺-Binding to Osteocalcin. Calcif Tissue Int 69, 337-342 (2001).
18. Lian, J. et al. Structure of the rat osteocalcin gene and regulation of vitamin D-dependent expression. Proc. Natl. Acad. Sci. USA 86, 1143-1147 (1989).
19. Hauschka, P. V. & Reid, M. L. Timed Appearance of a Calcium-Binding Protein Containing γ-Carboxyglutamic Acid in Developing Chick Bone. Dev Biol 65, 431-436 (1978).
20. Price, P.A., Williamson, M. K., Haba, T., Dell, R. B. & Jee, W. S. S. Excessive mineralization with growth plate closure in rats on chronic warfarin treatment. Proc. Natl. Acad. Sci. USA 79, 7734-7738 (1982).
21. Duey, P. et al. Increased bone formation in osteocalcin-deficient mice. Nature 382, 448-452 (1996).
22. Bodine, P. V. & Komm, B. S. Evidence that conditionally immortalized human osteoblasts express an osteocalcin receptor. Bone 25, 535-543 (1999).
23. Chenu, C. et al. Osteocalcin induces chemotaxis, secretion of matrix proteins, and calcium-mediated intracellular signaling in human osteoclast-like cells. J Cell Biol 127, 1149-1158 (1994).
24. Mundy, G. R. & Poser, J. W. Chemotactic activity of the gamma-carboxyglutamic acid-containing protein in bone. Calcif. Tissue Int 35, 164-168 (1983).
25. Wang, B.-C. Resolution of phase ambiguity in macromolecular crystallography. in Methods Enzymol Vol. 115 (eds Wychoff, H. W., Hirs, C. H. W. & Timasheff, S. N.) 90-112 (Academic Press, Orlando, 1985).
26. Hohm, L. & Sander, C. Protein structure comparison by alignment of distance matrices. J. Mol. Biol. 233, 123-138 (1993).
27. Kay, M. I., Young, R. A. & Posner, A. S. The crystal structure of hydroxyapatite. Nature 204, 1050-1052 (1964).
28. Klein, C. & Hurlbut, C. S. Manual of Mineralogy, (Wiley, New York, 1999).
29. Onuma, K., Ito, A., Tateishi, T. & Kameyama, T. Growth kinetics of hydroxyapatite crystal revealed by atomic force microscopy. J. Cryst. Growth 154, 118-125 (1995).
30. Eppell, S. J., Tong, W. & Katz, J. L. Shape and size of isolated bone mineralites measured using atomic force microscopy. J. Orthop. Res. 19, 1027-1034 (2001).
31. Calvo, M. S., Eyre, D. R. & Caren, M. G. Molecular Basis and Clinical Application of Biological Markers of Bone Turnover. Endocr Rev 17, 333-368 (1996).
32. Davies, P. L., Baardsnes, J., Kuiper, M. J. & Walker, V. K. Structure and function of antifreeze proteins. Philos Trans R Soc Lond B Biol Sci 357, 927-935 (2002).
33. Yang, D. S. et al. Identification of the ice-binding surface on a type III antifreeze protein with a “flatness function” algorithm. Biophys J 74, 2142-2151 (1998).
34. Tatusova, e.a. FEMS Microbiol Lett 174, 247-250 (1999).
35. Lattman, E. Use of the Rotation and Translation Functions. in Meth. Enzymol. Vol. 115 55-77, 1985).
36. Rossman, M. G. The Molecular Replacement Method. in Int. Sci. Rev. Ser. Vol. 13 (Gordon &Breach, New York, 1972).
37. Blundell, T. L. & Johnson, N. L. Protein Crystallography, (Academic Press, 1976).
38. Noren, C. J. e.a. Science 244, 182-188 (1989).
39. Goodford, P. J. J. Med. Chem. 28, 849-857 (1985).
40. Miranker, A. e.a. Proteins: Struct. Funct. Gen. 11, 29-34 (1991).
41. Goodsell, D. S. e.a. Proteins: Struct. Funct. Genet. 8, 195-202 (1990).
42. Kuntz, I.D. J. Mol. Biol. 161, 269-288 (1982).
43. Bartlett, P. A. e.a. Molecular Recognition in Chemical and Biological Problems, 182-196 (Special Publ., Royal Chem. Soc., 1989).
44. Lauri, G. e.a. J. Comput. Aided Mol. Des. 8, 51-66 (1994).
45. Martin, Y. C. J. Med. Cham. 35, 2145-2154 (1992).
46. Eisen, M. B. e.a. Proteins: Struc. Funct., Genet. 19, 199-221 (1994).
47. Bohm, H. J. J. Comp. Aid. Molec. Design. 6, 61-78 (1992).
48. Nishibata, Y. e.a. Tetrahedron 47, 8985 (1991).
49. Gillet, V. e.a. J. Comput. Aided Mol. Design. 7, 127-153 (1993).
50. Meng, E. C. e.a. J. Comp. Chem. 13, 505-525 (1992).
51. Travis, J. Science 262, 1374 (1993).
52. Robert, R. Conn's Current Therapy. (W.B. Saunders Company, USA, 1995).
53. Colombo, G., Fanti, P., Yao, C. & Halluche, H. H. Isolation and complete amino acid sequence of osteocalcin from canine bone. J Bone Miner Res 8, 733-743 (1993).
54. McPherson, A. Preparation and analysis of protein crystals, (Krieger Publishing Co., Malabar, 1989).
55. Otwinowski, Z. & Minor, W. Processing of X-ray diffraction data collected in oscillation mode. Methods Enzymol 276, 307-326 (1997).
56. Terwilliger, T. C. & Berendzen, J. Automated MAD and MIR structure solution. Acta Crystallogr D 55, 849-861 (1999).
57. Terwilliger, T. C. Maximum-likelihood density modification. Acta Crystallogr D 56, 1863-1871 (1999).
58. Jones, T. A., Zou, J. Y., Cowan, S. W. & Kjeldgaard. Improved methods for building protein models in electron density maps and the location of errors in these models. Acta Crystallogr A 47, 110-119 (1991).
59. Brunger, A. T. et al. Crystallography & NMR system: a new software suite for macromolecular structure determination. Acta Crystallogr D 54, 905-921 (1998).
60. Read, R. J. Improved Fourier coefficients for maps using phases from partial structures with errors. Acta. Cryst. A42, 140-149 (1986).

1. A computer-implemented method of identifying a compound that reduces osteocalcin activity, comprising
providing a computer program for execution on a computer, wherein the computer program, when executing on the computer, generates a 3D structure comprising i) amino acids 13-34 of SEQ ID NO: 5 of osteocalcin and ii) the structural coordinates in Table 3 corresponding to amino acids 13-34 of SEQ ID NO: 5;
designing a compound to mimic the 3D structure;
obtaining or synthesizing the compound and determining the ability of the compound to compete with osteocalcin for binding to hydroxyapatite in an assay, wherein reduced binding of osteocalcin to hydroxyapatite in the presence of the compound in the assay indicates that the compound reduces osteocalcin activity.

2. The method of claim 1, wherein designing the compound comprises comparing the structural coordinates of the compound to the structural coordinates of the 3D structure and determining whether the compound is spatially similar to the 3D structure and expected to competitively inhibit osteocalcin binding to hydroxyapatite.

3. The method of claim 1, wherein the 3D structure comprises a structurally equivalent sequence with 60% sequence identity to amino acids 13-34 of SEQ ID NO:5 and an Arg at position 19.

4. The method of claim 3, wherein the 3D structure corresponds to a 3D structure of a fragment of a pig or a human osteocalcin.

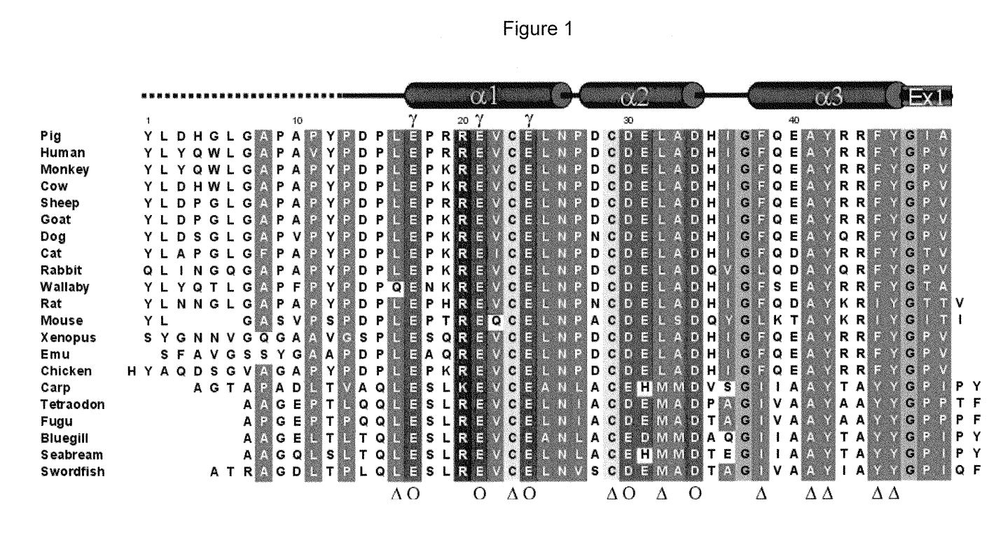

5. The method of claim 1, wherein the compound lacks at least one of the gamma-carboxylic acids corresponding to residues Gla17, Gla21 and Gla24 of SEQ ID NO:5 or a gamma-carboxylic acid on a Gla in FIG. 1 that is aligned with Gla17, Gla21 or Gla24 of SEQ ID NO:5.

6. The method of claim 1, wherein the compound comprises amino acids selected from the group consisting of
a. Gla17, Gla21 and Gla24 of SEQ ID NO:5; and
b. Pro13 to Asn27 of SEQ ID NO:5

7. The method of claim 1, wherein the structure comprises a conserved surface with a crystal structure which consists of 5 or less metal ions.

8. The method of claim 7, wherein the metal ions are calcium.

9. The method of claim 1, wherein the assay comprises an in vitro assay.

10. The method of claim 9, wherein the in vitro assay comprises:
a) incubating a test sample comprising (i) osteocalcin, (ii) the compound, and (iii) a substrate comprising hydroxyapatite;
b) detecting osteocalcin binding to hydroxyapatite.


SEQUENCE LISTING (21 sequences)

SEQ ID NO: 1 - protein, 49 aa, Macaca fascicularis
  MISC_FEATURE (17)..(17): Xaa = Glu modified to 3-gamma-carboxylated glutamic acid
YLYQWLGAPAPYPDPLXPKRXVCXLNPDCDELADHIGFQEAYRRFYGPV

SEQ ID NO: 2 - protein, 49 aa, Oryctolagus cuniculus
  MISC_FEATURE (17)..(17): Xaa = Glu modified to 3-gamma-carboxylated glutamic acid
QLINGQGAPAPYPDPLXPKRXVCXLNPDCDELADQVGLQDAYQRFYGPV

SEQ ID NO: 3 - protein, 49 aa, Homo sapiens
  MISC_FEATURE (17)..(17): Xaa = Glu modified to 3-gamma-carboxylated glutamic acid
YLYQWLGAPAVYPDPLXPRRXVCXLNPDCDELADHIGFQEAYRRFYGPV

SEQ ID NO: 4 - protein, 49 aa, Bos taurus
  MISC_FEATURE (17)..(17): Xaa = Glu modified to 3-gamma-carboxylated glutamic acid
YLDHWLGAPAPYPDPLXPKRXVCXLNPDCDELADHIGFQEAYRRFYGPV

SEQ ID NO: 5 - protein, 49 aa, Sus scrofa
  MISC_FEATURE (17)..(17): Xaa = Glu modified to 3-gamma-carboxylated glutamic acid
YLDHGLGAPAPYPDPLXPRRXVCXLNPDCDELADHIGFQEAYRRFYGIA

SEQ ID NO: 6 - protein, 49 aa, Ovis aries
  MISC_FEATURE (17)..(17): Xaa = Glu modified to 3-gamma-carboxylated glutamic acid
YLDPGLGAPAPYPDPLXPRRXVCXLNPDCDELADHIGFQEAYRRFYGPV

SEQ ID NO: 7 - protein, 49 aa, Capra hircus
  MISC_FEATURE (17)..(17): Xaa = Glu modified to 3-gamma-carboxylated glutamic acid
YLDPGLGAPAPYPDPLXPKRXVCXLNPDCDELADHIGFQEAYRRFYGPV

SEQ ID NO: 8 - protein, 49 aa, Canis familiaris
  MISC_FEATURE (17)..(17): Xaa = Glu modified to 3-gamma-carboxylated glutamic acid
YLDSGLGAPVPYPDPLXPKRXVCXLNPNCDELADHIGFQEAYQRFYGPV

SEQ ID NO: 9 - protein, 49 aa, Felis catus
  MISC_FEATURE (17)..(17): Xaa = Glu modified to 3-gamma-carboxylated glutamic acid
YLAPGLGFPAPYPDPLXPKRXICXLNPDCDELADHIGFQDAYRRFYGTV

SEQ ID NO: 10 - protein, 49 aa, Marcopus sp.
  MISC_FEATURE (17)..(17): Xaa = Glu modified to 3-gamma-carboxylated glutamic acid
YLYQTLGAPFPYPDPQXNKRXVCXLNPDCDELADHIGFSEAYRRFYGTA

SEQ ID NO: 11 - protein, 50 aa, Rattus rattus
  MISC_FEATURE (17)..(17): Xaa = Glu modified to 3-gamma-carboxylated glutamic acid
YLNNGLGAPAPYPDPLXPHRXVCXLNPNCDELADHIGFQDAYKRIYGTTV

SEQ ID NO: 12 - protein, 46 aa, Mus musculus
  MISC_FEATURE (13)..(13): Xaa = Glu modified to 3-gamma-carboxylated glutamic acid
YLGASVPSPDPLXPTRXQCXLNPACDELSDQYGLKTAYKRIYGITI

SEQ ID NO: 13 - protein, 49 aa, Xenopus sp.
  MISC_FEATURE (17)..(17): Xaa = Glu modified to 3-gamma-carboxylated glutamic acid
SYGNNVGQGAAVGSPLXSQRXVCXLNPDCDELADHIGFQEAYRRFYGPV

SEQ ID NO: 14 - protein, 48 aa, Dromaius novaehollandiae
  MISC_FEATURE (16)..(16): Xaa = Glu modified to 3-gamma-carboxylated glutamic acid
SFAVGSSYGAAPDPLXAQRXVCXLNPDCDELADHIGFQEAYRRFYGPV

SEQ ID NO: 15 - protein, 50 aa, Gallus gallus
  MISC_FEATURE (18)..(18): Xaa = Glu modified to 3-gamma-carboxylated glutamic acid
HYAQDSGVAGAPYPDPLXPKRXVCXLNPDCDELADHIGFQEAYRRFYGPV

SEQ ID NO: 16 - protein, 47 aa, Cyprinus carpio
  MISC_FEATURE (14)..(14): Xaa = Glu modified to 3-gamma-carboxylated glutamic acid
AGTAPADLTVAQLXSLKXVCXANLACEHMMDVSGIIAAYTAYGPIPY

SEQ ID NO: 17 - protein, 45 aa, Tetraodon sp.
  MISC_FEATURE (11)..(11): Xaa = Glu modified to 3-gamma-carboxylated glutamic acid
AAGEPTLQQLXSLRXVCXLNIACDEMADPAGIVAAYAAYYGPPTF

SEQ ID NO: 18 - protein, 45 aa, Fugu rubripes
  MISC_FEATURE (11)..(11): Xaa = Glu modified to 3-gamma-carboxylated glutamic acid
APGEPTPQQLXSLRXVCXLNIACDEMADTAGIVAAYAAYYGPPPF

SEQ ID NO: 19 - protein, 45 aa, Lepomis macrochirus
  MISC_FEATURE (11)..(11): Xaa = Glu modified to 3-gamma-carboxylated glutamic acid
AAGELTLTQLXSLRXVCXANLACEDMMDAQGIIAAYTAYYGPIPY

SEQ ID NO: 20 - protein, 45 aa, Pagellus sp.
  MISC_FEATURE (11)..(11): Xaa = Glu modified to 3-gamma-carboxylated glutamic acid
AAGQLSLTQLXSLRXVCXLNLACEHMMDTEGIIAAYTAYYGPIPY

SEQ ID NO: 21 - protein, 47 aa, Xiphias gladius
  MISC_FEATURE (13)..(13): Xaa = Glu modified to 3-gamma-carboxylated glutamic acid
ATRAGDLTPLQLXSLRXVCXLNVSCDEMADTAGIVAAYIAYYGPIQF